SEQUENCE LISTING (67 sequences)

SEQ ID NO: 1 - protein, 204 aa, Artificial Sequence
  SK_RBD
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 2 - protein, 18 aa, Artificial Sequence
  human_albumin_SP
MKWVTFISLLFLFSSAYS

SEQ ID NO: 3 - protein, 15 aa, Artificial Sequence
  Tetanus Toxoid Epitope-P2 domain
QYIKANSKFIGITEL

SEQ ID NO: 4 - protein, 27 aa, Artificial Sequence
  Foldon domain
GYIPEAPRDGQAYVRKDGEWVLLSTFL

SEQ ID NO: 5 - protein, 222 aa, Artificial Sequence
  SP-SK_RBD
MKWVTFISLLFLFSSAYSRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 6 - protein, 225 aa, Artificial Sequence
  RBD-ex1
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNG

SEQ ID NO: 7 - protein, 271 aa, Artificial Sequence
  RBD-ex2
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 8 - protein, 217 aa, Artificial Sequence
  RBD-ex3
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 9 - protein, 224 aa, Artificial Sequence
  SK_RBD-P2
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGSGSGQYIKANSKFIGITEL

SEQ ID NO: 10 - protein, 245 aa, Artificial Sequence
  RBD-ex1-P2
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGGSGSGQYIKANSKFIGITEL

SEQ ID NO: 11 - protein, 291 aa, Artificial Sequence
  RBD-ex2-P2
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSGSGSGQYIKANSKFIGITEL

SEQ ID NO: 12 - protein, 237 aa, Artificial Sequence
  RBD-ex3-P2
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSGSGQYIKANSKFIGITEL

SEQ ID NO: 13 - protein, 269 aa, Artificial Sequence
  RBD-Foldon-P2
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSGGSGYIPEAPRDGQAYVRKDGEWVLLSTFLGSGSGQYIKANSKFIGITEL

SEQ ID NO: 14 - DNA, 669 bp, Artificial Sequence
  SK_RBD_BEVS
atgaagtgggtgaccttcatcagcctgctgttcctgttctccagcgcctactctaggttcccaaacatcaccaacctgtgccctttcggagaggtgttcaacgctactagattcgccagcgtctacgcttggaaccgcaagcgtatcagcaactgcgtcgccgactactctgtgctgtacaactctgcttcattctccactttcaagtgctacggtgtcagccctaccaagctgaacgacctgtgcttcactaacgtctacgccgactctttcgtgatccgcggcgacgaagtccgtcagatcgctcctggtcagaccggaaagatcgctgactacaactacaagctgccagacgacttcactggttgcgtgatcgcttggaactcaaacaacctggactccaaggtcggtggcaactacaactacctgtacaggctgttcagaaagtccaacctgaagcctttcgagcgcgacatctcaaccgaaatctaccaggccggttccaccccctgcaacggtgtggagggcttcaactgctacttccccctgcaatcatacggtttccagccaaccaacggagtcggttaccagccttaccgcgtggtcgtgctgtccttcgaactgctccacgctcctgctactgtgtgcggccccaagaagtcaacttaa

SEQ ID NO: 15 - DNA, 669 bp, Artificial Sequence
  SK_RBD_CHO
atgaagtgggtcactttcatcagcctgttgtttctgttcagctccgcctactctagattcccaaacatcaccaatctgtgccccttcggcgaggtgtttaacgccacacgctttgcttccgtgtatgcctggaacaggaagcggatctctaattgcgtggctgactattccgtgctgtacaattccgccagcttctctacctttaagtgctatggcgtgtccccaaccaagctgaacgacctgtgcttcacaaacgtgtacgctgacagctttgtgatcaggggcgatgaggtgcggcagatcgctcctggccagaccggcaagatcgccgactacaactataagctgccagacgatttcacaggctgcgtgatcgcctggaactccaacaatctggatagcaaagtgggcggcaactacaattatctgtacagactgttccgcaagagcaacctgaagccctttgagagggacatcagcaccgaaatctaccaggctggctctacaccttgcaacggcgtggagggcttcaattgttattttcctctccagtcttacggcttccagccaacaaatggcgtgggctatcagccctacagggtggtggtgctgtcttttgagctgctgcacgctccagctaccgtgtgcggccctaagaagtccacatga

SEQ ID NO: 16 - DNA, 729 bp, Artificial Sequence
  SK_RBD-P2_BEVS
atgaagtgggtgaccttcatcagcctgctgttcctgttctccagcgcctactctaggttcccaaacatcaccaacctgtgccctttcggagaggtgttcaacgctactagattcgccagcgtctacgcttggaaccgcaagcgtatcagcaactgcgtcgccgactactctgtgctgtacaactctgcttcattctccactttcaagtgctacggtgtcagccctaccaagctgaacgacctgtgcttcactaacgtctacgccgactctttcgtgatccgcggcgacgaagtccgtcagatcgctcctggtcagaccggaaagatcgctgactacaactacaagctgccagacgacttcactggttgcgtgatcgcttggaactcaaacaacctggactccaaggtcggtggcaactacaactacctgtacaggctgttcagaaagtccaacctgaagcctttcgagcgcgacatctcaaccgaaatctaccaggccggttccaccccctgcaacggtgtggagggcttcaactgctacttccccctgcaatcatacggtttccagccaaccaacggagtcggttaccagccttaccgcgtggtcgtgctgtccttcgaactgctccacgctcctgctactgtgtgcggccccaagaagtcaactggcagcggatctggacagtacatcaaggctaactccaagttcatcggaatcactgagctgtaa

SEQ ID NO: 17 - DNA, 729 bp, Artificial Sequence
  SK_RBD-P2_CHO
atgaagtgggtcactttcatcagcctgttgtttctgttcagctccgcctactctagattcccaaacatcaccaatctgtgccccttcggcgaggtgtttaacgccacacgctttgcttccgtgtatgcctggaacaggaagcggatctctaattgcgtggctgactattccgtgctgtacaattccgccagcttctctacctttaagtgctatggcgtgtccccaaccaagctgaacgacctgtgcttcacaaacgtgtacgctgacagctttgtgatcaggggcgatgaggtgcggcagatcgctcctggccagaccggcaagatcgccgactacaactataagctgccagacgatttcacaggctgcgtgatcgcctggaactccaacaatctggatagcaaagtgggcggcaactacaattatctgtacagactgttccgcaagagcaacctgaagccctttgagagggacatcagcaccgaaatctaccaggctggctctacaccttgcaacggcgtggagggcttcaattgttattttcctctccagtcttacggcttccagccaacaaatggcgtgggctatcagccctacagggtggtggtgctgtcttttgagctgctgcacgctccagctaccgtgtgcggccctaagaagtccacaggctccggctccggccagtacatcaaggccaactccaagttcatcggcatcaccgagctgtaa

SEQ ID NO: 18 - DNA, 792 bp, Artificial Sequence
  RBD-ex1-P2_BEVS
atgaagtgggtgactttcatctccctgctgttcctgttctccagcgcttacagccagcctaccgaatcaatcgtccgtttcccaaacatcactaacctgtgccctttcggagaggtgttcaacgccaccagattcgcttccgtctacgcctggaaccgcaagcgtatctctaactgcgtcgctgactactcagtgctgtacaacagcgcctctttctcaaccttcaagtgctacggagtgtctcctactaagctgaacgacctgtgcttcaccaacgtctacgctgactcattcgtgatccgcggtgacgaggtccgtcagatcgctcccggacagactggcaagatcgccgactacaactacaagctgccagacgacttcaccggttgcgtgatcgcctggaactctaacaacctggactcaaaggtcggtggcaactacaactacctgtacaggctgttcagaaagtctaacctgaagcctttcgagcgcgacatctccactgaaatctaccaggctggtagcaccccctgcaacggcgtggaaggattcaactgctacttccctctgcaatcatacggcttccagcccactaacggcgtcggataccagccataccgtgtggtcgtgctgtccttcgagctgctccacgctcctgctactgtgtgcggccccaagaagagcaccaacctggtcaagaacaagtgcgtgaacttcaacttcaacggaggctccggaagcggacagtacatcaaggccaacagcaagttcatcggtatcaccgagctgtaa

SEQ ID NO: 19 - DNA, 792 bp, Artificial Sequence
  RBD-ex1-P2_CHO
atgaagtgggtcactttcatcagcctgttgtttctgttcagctccgcctactctcagcccaccgagtccatcgtgagattcccaaacatcaccaatctgtgccccttcggcgaggtgtttaacgccacacgctttgcttccgtgtatgcctggaacaggaagcggatctctaattgcgtggctgactattccgtgctgtacaattccgccagcttctctacctttaagtgctatggcgtgtccccaaccaagctgaacgacctgtgcttcacaaacgtgtacgctgacagctttgtgatcaggggcgatgaggtgcggcagatcgctcctggccagaccggcaagatcgccgactacaactataagctgccagacgatttcacaggctgcgtgatcgcctggaactccaacaatctggatagcaaagtgggcggcaactacaattatctgtacagactgttccgcaagagcaacctgaagccctttgagagggacatcagcaccgaaatctaccaggctggctctacaccttgcaacggcgtggagggcttcaattgttattttcctctccagtcttacggcttccagccaacaaatggcgtgggctatcagccctacagggtggtggtgctgtcttttgagctgctgcacgctccagctaccgtgtgcggccctaagaagtccacaaatctggtgaagaacaagtgcgtgaacttcaacttcaacggcggctccggctccggccagtacatcaaggccaactccaagttcatcggcatcaccgagctgtaa

SEQ ID NO: 20 - DNA, 930 bp, Artificial Sequence
  RBD-ex2-P2_BEVS
atgaagtgggtgactttcatctccctgctgttcctgttctccagcgcttacagccagcctaccgaatcaatcgtccgtttcccaaacatcactaacctgtgccctttcggagaggtgttcaacgccaccagattcgcttccgtctacgcctggaaccgcaagcgtatctctaactgcgtcgctgactactcagtgctgtacaacagcgcctctttctcaaccttcaagtgctacggagtgtctcctactaagctgaacgacctgtgcttcaccaacgtctacgctgactcattcgtgatccgcggtgacgaggtccgtcagatcgctcccggacagactggcaagatcgccgactacaactacaagctgccagacgacttcaccggttgcgtgatcgcctggaactctaacaacctggactcaaaggtcggtggcaactacaactacctgtacaggctgttcagaaagtctaacctgaagcctttcgagcgcgacatctccactgaaatctaccaggctggtagcaccccctgcaacggcgtggaaggattcaactgctacttccctctgcaatcatacggcttccagcccactaacggcgtcggataccagccataccgtgtggtcgtgctgtccttcgagctgctccacgctcctgctactgtgtgcggccccaagaagagcaccaacctggtcaagaacaagtgcgtgaacttcaacttcaacggactgaccggtactggcgtgctgaccgaatccaacaagaagttcctgcctttccagcagttcggtcgcgacatcgctgacaccactgacgccgtccgtgaccctcagaccctggagatcctggacatcactccctgctccggctccggaagcggacagtacatcaaggccaacagcaagttcatcggtatcaccgagctgtaa

SEQ ID NO: 21 - DNA, 930 bp, Artificial Sequence
  RBD-ex2-P2_CHO
atgaagtgggtcactttcatcagcctgttgtttctgttcagctccgcctactctcagcccaccgagtccatcgtgagattcccaaacatcaccaatctgtgccccttcggcgaggtgtttaacgccacacgctttgcttccgtgtatgcctggaacaggaagcggatctctaattgcgtggctgactattccgtgctgtacaattccgccagcttctctacctttaagtgctatggcgtgtccccaaccaagctgaacgacctgtgcttcacaaacgtgtacgctgacagctttgtgatcaggggcgatgaggtgcggcagatcgctcctggccagaccggcaagatcgccgactacaactataagctgccagacgatttcacaggctgcgtgatcgcctggaactccaacaatctggatagcaaagtgggcggcaactacaattatctgtacagactgttccgcaagagcaacctgaagccctttgagagggacatcagcaccgaaatctaccaggctggctctacaccttgcaacggcgtggagggcttcaattgttattttcctctccagtcttacggcttccagccaacaaatggcgtgggctatcagccctacagggtggtggtgctgtcttttgagctgctgcacgctccagctaccgtgtgcggccctaagaagtccacaaatctggtgaagaacaagtgcgtgaacttcaacttcaacggcctgaccggcacaggcgtgctgaccgagtccaataagaagttcctgccctttcagcagttcggcagagacatcgccgataccacagacgctgtgcgcgatccccagaccctggagatcctggacatcacaccttgcagcggctccggctccggccagtacatcaaggccaactccaagttcatcggcatcaccgagctgtaa

SEQ ID NO: 22 - DNA, 768 bp, Artificial Sequence
  RBD-ex3-P2_BEVS
atgaagtgggtgactttcatctccctgctgttcctgttctccagcgcttacagccagcctaccgaatcaatcgtccgtttcccaaacatcactaacctgtgccctttcggagaggtgttcaacgccaccagattcgcttccgtctacgcctggaaccgcaagcgtatctctaactgcgtcgctgactactcagtgctgtacaacagcgcctctttctcaaccttcaagtgctacggagtgtctcctactaagctgaacgacctgtgcttcaccaacgtctacgctgactcattcgtgatccgcggtgacgaggtccgtcagatcgctcccggacagactggcaagatcgccgactacaactacaagctgccagacgacttcaccggttgcgtgatcgcctggaactctaacaacctggactcaaaggtcggtggcaactacaactacctgtacaggctgttcagaaagtctaacctgaagcctttcgagcgcgacatctccactgaaatctaccaggctggtagcaccccctgcaacggcgtggaaggattcaactgctacttccctctgcaatcatacggcttccagcccactaacggcgtcggataccagccataccgtgtggtcgtgctgtccttcgagctgctccacgctcctgctactgtgtgcggccccaagaagagcaccaacctggtcaagaacaagggctccggaagcggacagtacatcaaggccaacagcaagttcatcggtatcaccgagctgtaa

SEQ ID NO: 23 - DNA, 768 bp, Artificial Sequence
  RBD-ex3-P2_CHO
atgaagtgggtcactttcatcagcctgttgtttctgttcagctccgcctactctcagcccaccgagtccatcgtgagattcccaaacatcaccaatctgtgccccttcggcgaggtgtttaacgccacacgctttgcttccgtgtatgcctggaacaggaagcggatctctaattgcgtggctgactattccgtgctgtacaattccgccagcttctctacctttaagtgctatggcgtgtccccaaccaagctgaacgacctgtgcttcacaaacgtgtacgctgacagctttgtgatcaggggcgatgaggtgcggcagatcgctcctggccagaccggcaagatcgccgactacaactataagctgccagacgatttcacaggctgcgtgatcgcctggaactccaacaatctggatagcaaagtgggcggcaactacaattatctgtacagactgttccgcaagagcaacctgaagccctttgagagggacatcagcaccgaaatctaccaggctggctctacaccttgcaacggcgtggagggcttcaattgttattttcctctccagtcttacggcttccagccaacaaatggcgtgggctatcagccctacagggtggtggtgctgtcttttgagctgctgcacgctccagctaccgtgtgcggccctaagaagtccacaaatctggtgaagaacaagggctccggctccggccagtacatcaaggccaactccaagttcatcggcatcaccgagctgtaa

SEQ ID NO: 24 - DNA, 864 bp, Artificial Sequence
  RBD-Foldon-P2_BEVS
atgaagtgggtgactttcatctccctgctgttcctgttctccagcgcttacagccagcctaccgaatcaatcgtccgtttcccaaacatcactaacctgtgccctttcggagaggtgttcaacgccaccagattcgcttccgtctacgcctggaaccgcaagcgtatctctaactgcgtcgctgactactcagtgctgtacaacagcgcctctttctcaaccttcaagtgctacggagtgtctcctactaagctgaacgacctgtgcttcaccaacgtctacgctgactcattcgtgatccgcggtgacgaggtccgtcagatcgctcccggacagactggcaagatcgccgactacaactacaagctgccagacgacttcaccggttgcgtgatcgcctggaactctaacaacctggactcaaaggtcggtggcaactacaactacctgtacaggctgttcagaaagtctaacctgaagcctttcgagcgcgacatctccactgaaatctaccaggctggtagcaccccctgcaacggcgtggaaggattcaactgctacttccctctgcaatcatacggcttccagcccactaacggcgtcggataccagccataccgtgtggtcgtgctgtccttcgagctgctccacgctcctgctactgtgtgcggccccaagaagagcaccaacctggtcaagaacaagggaagcggtggctccggttacatccctgaagctccccgcgacggacaggcctacgtccgtaaggacggagagtgggtgctgctgtcaactttcctgggatctggttcaggccagtacatcaaggctaactccaagttcatcggtatcaccgaactgtaa

SEQ ID NO: 25 - DNA, 864 bp, Artificial Sequence
  RBD-Foldon-P2_CHO
atgaagtgggtcactttcatcagcctgttgtttctgttcagctccgcctactctcagcccaccgagtccatcgtgagattcccaaacatcaccaatctgtgccccttcggcgaggtgtttaacgccacacgctttgcttccgtgtatgcctggaacaggaagcggatctctaattgcgtggctgactattccgtgctgtacaattccgccagcttctctacctttaagtgctatggcgtgtccccaaccaagctgaacgacctgtgcttcacaaacgtgtacgctgacagctttgtgatcaggggcgatgaggtgcggcagatcgctcctggccagaccggcaagatcgccgactacaactataagctgccagacgatttcacaggctgcgtgatcgcctggaactccaacaatctggatagcaaagtgggcggcaactacaattatctgtacagactgttccgcaagagcaacctgaagccctttgagagggacatcagcaccgaaatctaccaggctggctctacaccttgcaacggcgtggagggcttcaattgttattttcctctccagtcttacggcttccagccaacaaatggcgtgggctatcagccctacagggtggtggtgctgtcttttgagctgctgcacgctccagctaccgtgtgcggccctaagaagtccacaaatctggtgaagaacaagggctccggcggctccggttacatccctgaagctccccgcgacggacaggcctacgtccgtaaggacggagagtgggtgctgctgtcaactttcctgggctccggctccggccagtacatcaaggccaactccaagttcatcggcatcaccgagctgtaa

SEQ ID NO: 26 - protein, 418 aa, Artificial Sequence
  N protein of SARS-CoV-2
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 27 - protein, 436 aa, Artificial Sequence
  N protein of SARS-CoV-2 linked to Human albumin signal peptide
MKWVTFISLLFLFSSAYSSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 28 - DNA, 1311 bp, Artificial Sequence
  N protein_BEVS
atgaaatgggtcaccttcatcagtctgctgttcctgttctcttccgcttactcctccgacaacggtcctcaaaaccaacgcaacgcaccccgcatcaccttcggtggcccaagcgactctactggttccaaccagaacggtgaacgctcaggcgctcgttccaagcagcgccgtccacagggcctgcctaacaacaccgcttcctggttcaccgccctgactcagcacggaaaggaggacctgaagttccctcgtggacagggtgtgcccatcaacaccaactccagccctgacgaccagatcggatactacaggagagccactcgccgtatcaggggaggtgacggcaagatgaaggacctgtcccccagatggtacttctactacctcggcaccggacccgaggctggactgccatacggtgccaacaaggacggtatcatctgggtggctaccgaaggcgccctgaacactcccaaggaccacatcggtactaggaacccagctaacaacgctgccatcgtcctgcaactgccacagggcaccactctgcctaagggtttctacgctgaaggcagccgcggcggatctcaggcctcttcacgttccagctctcgctcccgtaactcatccaggaacagcaccccaggcagctctaggggaacttctcctgctagaatggctggaaacggtggcgacgctgccctggctctgctgctgctggacagactgaaccagctggagagcaagatgtctggcaagggacagcagcagcagggacagactgtgaccaagaagtccgctgctgaggcttccaagaagcccaggcagaagagaaccgctactaaggcctacaacgtcacccaggccttcggaaggagaggtccagagcagactcagggcaacttcggtgaccaggaactgatccgccagggcaccgactacaagcactggcctcagatcgctcagttcgccccctcagcttccgccttcttcggaatgtctcgtatcggtatggaagtgaccccatcaggcacttggctgacctacactggagctatcaagctggatgacaaggaccctaacttcaaggaccaggtcatcctgctgaacaagcacatcgacgcctacaagaccttccctcccactgagcctaagaaggacaagaagaagaaggctgacgaaacccaggccctgcctcagcgccagaagaagcagcagactgtcactctgctgcccgctgccgacctggacgacttcagcaagcagctgcaacagtctatgtcatccgctgactcaactcaggcctaa

SEQ ID NO: 29 - DNA, 1311 bp, Artificial Sequence
  N protein_CHO
atgaagtgggtcactttcatcagcctgttgtttctgttcagctccgcctactcttcagataacggtccacagaaccagcggaatgctcccagaatcaccttcggcggtccaagcgactcaacaggcagtaaccagaacggcgagcggtccggcgctagatccaagcagagacggcctcagggcctgccaaacaacaccgcctcttggtttaccgctctgacccagcacggcaaggaggacctgaagtttcccagaggccagggcgtgcccatcaataccaactccagcccagatgaccagatcggctattaccggagagccacaaggagaatccgcggcggcgacggcaagatgaaggacctgtccccacggtggtacttctactatctgggcaccggccccgaggctggcctgccttatggcgctaacaaggatggcatcatctgggtggctacagagggcgctctgaatacccctaaggatcacatcggcacaagaaatccagctaataacgccgctatcgtgctgcaactgccccagggcaccacactgccaaagggcttttacgctgagggctctcgcggcggctcccaggcttcttccagaagctcttccagatccagaaactcctctcgcaactctacccctggctcttccagaggcacaagccctgctagaatggccggcaatggcggcgacgccgctctggccctgctgctgctggataggctgaaccagctggagtccaagatgtctggcaagggccagcagcagcagggccagacagtgaccaagaagtctgccgctgaggcttccaagaagcctcggcagaagagaaccgccacaaaggcttataacgtgacccaggcttttggcagaagaggccctgagcagacccagggcaacttcggcgatcaggagctgatcagacagggcaccgattacaagcattggccacagatcgcccagtttgctccttccgccagcgccttctttggcatgtccaggatcggcatggaggtgacaccctctggcacctggctgacatataccggcgctatcaagctggacgataaggacccaaacttcaaggatcaggtaatcctgctgaacaagcacatcgacgcctacaagacattcccacctaccgagccaaagaaggacaagaagaagaaggccgatgaaacccaggccctgccccagagacagaagaagcagcagacagtgaccctgctgccagctgccgatctggacgatttctcaaaacagcttcagcagtcaatgtcatccgccgattcaactcaggcataa

SEQ ID NO: 30 - DNA, 3822 bp, Artificial Sequence
  Nucleotide sequence of the gene coding for the spike protein of SARS-CoV-2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 31 - DNA, 2058 bp, Artificial Sequence
  Nucleotide sequence of the gene coding for the S1 domain of spike protein of SARS-CoV-2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagt

SEQ ID NO: 32 - DNA, 1764 bp, Artificial Sequence
  Nucleotide sequence of the gene coding for the S2 domain of spike protein of SARS-CoV-2
gtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 33 - DNA, 582 bp, Artificial Sequence
  Nucleotide sequence of the gene coding for the RBD of spike protein of SARS-CoV-2
aatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtt

SEQ ID NO: 34 - protein, 1273 aa, Artificial Sequence
  Amino acid sequence of the spike protein of SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 35 - protein, 673 aa, Artificial Sequence
  Amino acid sequence of the S1 domain of spike protein of SARS-CoV-2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARS

SEQ ID NO: 36 - protein, 587 aa, Artificial Sequence
  Amino acid sequence of the S2 domain of spike protein of SARS-CoV-2
VASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 37 - protein, 194 aa, Artificial Sequence
  Amino acid sequence of the RBD of spike protein of SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 38 - DNA, 732 bp, Artificial Sequence
  RBD-ex1_BEVS
atgaagtgggtgactttcatctccctgctgttcctgttctccagcgcttacagccagcctaccgaatcaatcgtccgtttcccaaacatcactaacctgtgccctttcggagaggtgttcaacgccaccagattcgcttccgtctacgcctggaaccgcaagcgtatctctaactgcgtcgctgactactcagtgctgtacaacagcgcctctttctcaaccttcaagtgctacggagtgtctcctactaagctgaacgacctgtgcttcaccaacgtctacgctgactcattcgtgatccgcggtgacgaggtccgtcagatcgctcccggacagactggcaagatcgccgactacaactacaagctgccagacgacttcaccggttgcgtgatcgcctggaactctaacaacctggactcaaaggtcggtggcaactacaactacctgtacaggctgttcagaaagtctaacctgaagcctttcgagcgcgacatctccactgaaatctaccaggctggtagcaccccctgcaacggcgtggaaggattcaactgctacttccctctgcaatcatacggcttccagcccactaacggcgtcggataccagccataccgtgtggtcgtgctgtccttcgagctgctccacgctcctgctactgtgtgcggccccaagaagagcaccaacctggtcaagaacaagtgcgtgaacttcaacttcaacggataa

SEQ ID NO: 39 - DNA, 732 bp, Artificial Sequence
  RBD-ex1_CHO
atgaagtgggtcactttcatcagcctgttgtttctgttcagctccgcctactctcagcccaccgagtccatcgtgagattcccaaacatcaccaatctgtgccccttcggcgaggtgtttaacgccacacgctttgcttccgtgtatgcctggaacaggaagcggatctctaattgcgtggctgactattccgtgctgtacaattccgccagcttctctacctttaagtgctatggcgtgtccccaaccaagctgaacgacctgtgcttcacaaacgtgtacgctgacagctttgtgatcaggggcgatgaggtgcggcagatcgctcctggccagaccggcaagatcgccgactacaactataagctgccagacgatttcacaggctgcgtgatcgcctggaactccaacaatctggatagcaaagtgggcggcaactacaattatctgtacagactgttccgcaagagcaacctgaagccctttgagagggacatcagcaccgaaatctaccaggctggctctacaccttgcaacggcgtggagggcttcaattgttattttcctctccagtcttacggcttccagccaacaaatggcgtgggctatcagccctacagggtggtggtgctgtcttttgagctgctgcacgctccagctaccgtgtgcggccctaagaagtccacaaatctggtgaagaacaagtgcgtgaacttcaacttcaacggctaa

SEQ ID NO: 40 - DNA, 870 bp, Artificial Sequence
  RBD-ex2_BEVS
atgaagtgggtgactttcatctccctgctgttcctgttctccagcgcttacagccagcctaccgaatcaatcgtccgtttcccaaacatcactaacctgtgccctttcggagaggtgttcaacgccaccagattcgcttccgtctacgcctggaaccgcaagcgtatctctaactgcgtcgctgactactcagtgctgtacaacagcgcctctttctcaaccttcaagtgctacggagtgtctcctactaagctgaacgacctgtgcttcaccaacgtctacgctgactcattcgtgatccgcggtgacgaggtccgtcagatcgctcccggacagactggcaagatcgccgactacaactacaagctgccagacgacttcaccggttgcgtgatcgcctggaactctaacaacctggactcaaaggtcggtggcaactacaactacctgtacaggctgttcagaaagtctaacctgaagcctttcgagcgcgacatctccactgaaatctaccaggctggtagcaccccctgcaacggcgtggaaggattcaactgctacttccctctgcaatcatacggcttccagcccactaacggcgtcggataccagccataccgtgtggtcgtgctgtccttcgagctgctccacgctcctgctactgtgtgcggccccaagaagagcaccaacctggtcaagaacaagtgcgtgaacttcaacttcaacggactgaccggtactggcgtgctgaccgaatccaacaagaagttcctgcctttccagcagttcggtcgcgacatcgctgacaccactgacgccgtccgtgaccctcagaccctggagatcctggacatcactccctgctcctaa

SEQ ID NO: 41 - DNA, 870 bp, Artificial Sequence
  RBD-ex2_CHO
atgaagtgggtcactttcatcagcctgttgtttctgttcagctccgcctactctcagcccaccgagtccatcgtgagattcccaaacatcaccaatctgtgccccttcggcgaggtgtttaacgccacacgctttgcttccgtgtatgcctggaacaggaagcggatctctaattgcgtggctgactattccgtgctgtacaattccgccagcttctctacctttaagtgctatggcgtgtccccaaccaagctgaacgacctgtgcttcacaaacgtgtacgctgacagctttgtgatcaggggcgatgaggtgcggcagatcgctcctggccagaccggcaagatcgccgactacaactataagctgccagacgatttcacaggctgcgtgatcgcctggaactccaacaatctggatagcaaagtgggcggcaactacaattatctgtacagactgttccgcaagagcaacctgaagccctttgagagggacatcagcaccgaaatctaccaggctggctctacaccttgcaacggcgtggagggcttcaattgttattttcctctccagtcttacggcttccagccaacaaatggcgtgggctatcagccctacagggtggtggtgctgtcttttgagctgctgcacgctccagctaccgtgtgcggccctaagaagtccacaaatctggtgaagaacaagtgcgtgaacttcaacttcaacggcctgaccggcacaggcgtgctgaccgagtccaataagaagttcctgccctttcagcagttcggcagagacatcgccgataccacagacgctgtgcgcgatccccagaccctggagatcctggacatcacaccttgcagctaa

SEQ ID NO: 42 - DNA, 708 bp, Artificial Sequence
  RBD-ex3_BEVS
atgaagtgggtgactttcatctccctgctgttcctgttctccagcgcttacagccagcctaccgaatcaatcgtccgtttcccaaacatcactaacctgtgccctttcggagaggtgttcaacgccaccagattcgcttccgtctacgcctggaaccgcaagcgtatctctaactgcgtcgctgactactcagtgctgtacaacagcgcctctttctcaaccttcaagtgctacggagtgtctcctactaagctgaacgacctgtgcttcaccaacgtctacgctgactcattcgtgatccgcggtgacgaggtccgtcagatcgctcccggacagactggcaagatcgccgactacaactacaagctgccagacgacttcaccggttgcgtgatcgcctggaactctaacaacctggactcaaaggtcggtggcaactacaactacctgtacaggctgttcagaaagtctaacctgaagcctttcgagcgcgacatctccactgaaatctaccaggctggtagcaccccctgcaacggcgtggaaggattcaactgctacttccctctgcaatcatacggcttccagcccactaacggcgtcggataccagccataccgtgtggtcgtgctgtccttcgagctgctccacgctcctgctactgtgtgcggccccaagaagagcaccaacctggtcaagaacaagtaa

SEQ ID NO: 43 - DNA, 708 bp, Artificial Sequence
  RBD-ex3_CHO
atgaagtgggtcactttcatcagcctgttgtttctgttcagctccgcctactctcagcccaccgagtccatcgtgagattcccaaacatcaccaatctgtgccccttcggcgaggtgtttaacgccacacgctttgcttccgtgtatgcctggaacaggaagcggatctctaattgcgtggctgactattccgtgctgtacaattccgccagcttctctacctttaagtgctatggcgtgtccccaaccaagctgaacgacctgtgcttcacaaacgtgtacgctgacagctttgtgatcaggggcgatgaggtgcggcagatcgctcctggccagaccggcaagatcgccgactacaactataagctgccagacgatttcacaggctgcgtgatcgcctggaactccaacaatctggatagcaaagtgggcggcaactacaattatctgtacagactgttccgcaagagcaacctgaagccctttgagagggacatcagcaccgaaatctaccaggctggctctacaccttgcaacggcgtggagggcttcaattgttattttcctctccagtcttacggcttccagccaacaaatggcgtgggctatcagccctacagggtggtggtgctgtcttttgagctgctgcacgctccagctaccgtgtgcggccctaagaagtccacaaatctggtgaagaacaagtaa

SEQ ID NO: 44 - protein, 1273 aa, Unknown; identical to SEQ ID NO: 34
  Spike protein of B.1.429 variant

SEQ ID NO: 45 - protein, 1270 aa, Unknown
  Spike protein of B.1.1.7 variant
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 46 - protein, 1270 aa, Unknown
  Spike protein of B.1.351 variant
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 47 - protein, 1273 aa, Unknown
  Spike protein of B.1.1.248 variant
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 48 - protein, 1273 aa, Unknown
  Spike protein of B.1.429 variant
MFVFLVLLPLVSIQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 49 - DNA, 792 bp, Artificial Sequence
  Codon-optimized nucleic acid sequence of RBD ext1-P2 of variant B.1.1.7 for CHO expression system
atgaagtgggtgaccttcatctccctgctgttcctgttctcctccgcctatagccagccaaccgagtctatcgtgagattcccaaatatcacaaacctgtgccccttcggcgaggtgtttaatgccacccgctttgcctccgtgtacgcctggaataggaagcggatctctaactgcgtggctgactattccgtgctgtacaactccgcctccttctccaccttcaagtgctatggcgtgtcccccaccaagctgaatgacctgtgcttcacaaacgtgtacgctgacagctttgtgatcaggggcgatgaggtgcggcagatcgctcctggacagaccggcaacatcgccgactacaattataagctgccagacgacttcaccggctgcgtgatcgcctggaactccaacaatctggatagcaaagtgggcggcaactacaattatctgtacagactgttccgcaagagcaatctgaagccctttgagagggacatcagcaccgaaatctaccaggctggctctacaccttgcaatggcgtgaagggcttcaactgttattttcctctgcagtcttacggcttccagccaacctacggcgtgggctatcagccctacagggtggtggtgctgtcttttgagctgctgcacgctccagctaccgtgtgcggacctaagaagtccacaaatctggtgaagaacaagtgcgtgaacttcaacttcaacggcggctctggctccggccagtacatcaaggccaactctaagttcatcggcatcacagagctgtga

SEQ ID NO: 50 - DNA, 792 bp, Artificial Sequence; identical to SEQ ID NO: 49
  Codon-optimized nucleic acid sequence of RBD ext1-P2 of variant B.1.351 for CHO expression system

SEQ ID NO: 51 - DNA, 792 bp, Artificial Sequence
  Codon-optimized nucleic acid sequence of RBD ext1-P2 of variant B.1.1.248 for CHO expression system
atgaagtgggtgaccttcatctccctgctgttcctgttctcctccgcctatagccagccaaccgagtctatcgtgagattcccaaatatcacaaacctgtgccccttcggcgaggtgtttaatgccacccgctttgcctccgtgtacgcctggaataggaagcggatctctaactgcgtggctgactattccgtgctgtacaactccgcctccttctccaccttcaagtgctatggcgtgtcccccaccaagctgaatgacctgtgcttcacaaacgtgtacgctgacagctttgtgatcaggggcgatgaggtgcggcagatcgctcctggacagaccggcaccatcgccgactacaattataagctgccagacgacttcaccggctgcgtgatcgcctggaactccaacaatctggatagcaaagtgggcggcaactacaattatctgtacagactgttccgcaagagcaatctgaagccctttgagagggacatcagcaccgaaatctaccaggctggctctacaccttgcaatggcgtgaagggcttcaactgttattttcctctgcagtcttacggcttccagccaacctacggcgtgggctatcagccctacagggtggtggtgctgtcttttgagctgctgcacgctccagctaccgtgtgcggacctaagaagtccacaaatctggtgaagaacaagtgcgtgaacttcaacttcaacggcggctctggctccggccagtacatcaaggccaactctaagttcatcggcatcacagagctgtga

SEQ ID NO: 52 - DNA, 792 bp, Artificial Sequence
  Codon-optimized nucleic acid sequence of RBD ext1-P2 of variant B.1.429 for CHO expression system
atgaagtgggtgaccttcatctccctgctgttcctgttctcctccgcctatagccagccaaccgagtctatcgtgagattcccaaatatcacaaacctgtgccccttcggcgaggtgtttaatgccacccgctttgcctccgtgtacgcctggaataggaagcggatctctaactgcgtggctgactattccgtgctgtacaactccgcctccttctccaccttcaagtgctatggcgtgtcccccaccaagctgaatgacctgtgcttcacaaacgtgtacgctgacagctttgtgatcaggggcgatgaggtgcggcagatcgctcctggacagaccggcaagatcgccgactacaattataagctgccagacgacttcaccggctgcgtgatcgcctggaactccaacaatctggatagcaaagtgggcggcaactacaattatcggtacagactgttccgcaagagcaatctgaagccctttgagagggacatcagcaccgaaatctaccaggctggctctacaccttgcaatggcgtggagggcttcaactgttattttcctctgcagtcttacggcttccagccaaccaacggcgtgggctatcagccctacagggtggtggtgctgtcttttgagctgctgcacgctccagctaccgtgtgcggacctaagaagtccacaaatctggtgaagaacaagtgcgtgaacttcaacttcaacggcggctctggctccggccagtacatcaaggccaactctaagttcatcggcatcacagagctgtga

SEQ ID NO: 53 - DNA, 864 bp, Artificial Sequence
  Codon-optimized nucleic acid sequence of RBD ext3-foldon-P2 of variant B.1.1.7 for CHO expression system
atgaagtgggtgaccttcatcagcctgctgttcctgttctcctccgcctattcccagcctaccgagagcatcgtgaggttccctaacatcacaaatctgtgcccattcggcgaggtgtttaacgccacccggtttgcctccgtgtacgcctggaacaggaagcggatcagcaattgcgtggctgactattctgtgctgtacaattccgcctccttctccaccttcaagtgctatggcgtgagcccaaccaagctgaacgacctgtgcttcacaaacgtgtacgctgactcttttgtgatcaggggcgatgaggtgcggcagatcgctccaggacagaccggcaagatcgctgactacaactataagctgcctgacgacttcaccggctgcgtgatcgcctggaactccaacaatctggattccaaagtgggcggcaactacaattatctgtacagactgttccgcaagtctaacctgaagccatttgagagagacatctccaccgaaatctaccaggctggcagcacaccatgcaacggagtggagggcttcaattgttattttcccctgcagtcctacggcttccagcctacctacggcgtgggctatcagccataccgcgtggtggtgctgtcctttgagctgctgcacgctccagctaccgtgtgcggacccaagaagagcacaaacctggtgaagaataagggcagcggcggctctggctatatccccgaggctcctagagacggccaggcctacgtgcgcaaggatggcgagtgggtgctgctgtctaccttcctgggctctggctccggccagtacatcaaggccaactccaagtttatcggcatcacagagctgtga

SEQ ID NO: 54 - DNA, 864 bp, Artificial Sequence
  Codon-optimized nucleic acid sequence of RBD ext3-foldon-P2 of variant B.1.351 for CHO expression system
atgaagtgggtgaccttcatcagcctgctgttcctgttctcctccgcctattcccagcctaccgagagcatcgtgaggttccctaacatcacaaatctgtgcccattcggcgaggtgtttaacgccacccggtttgcctccgtgtacgcctggaacaggaagcggatcagcaattgcgtggctgactattctgtgctgtacaattccgcctccttctccaccttcaagtgctatggcgtgagcccaaccaagctgaacgacctgtgcttcacaaacgtgtacgctgactcttttgtgatcaggggcgatgaggtgcggcagatcgctccaggacagaccggcaacatcgctgactacaactataagctgcctgacgacttcaccggctgcgtgatcgcctggaactccaacaatctggattccaaagtgggcggcaactacaattatctgtacagactgttccgcaagtctaacctgaagccatttgagagagacatctccaccgaaatctaccaggctggcagcacaccatgcaacggagtgaagggcttcaattgttattttcccctgcagtcctacggcttccagcctacctacggcgtgggctatcagccataccgcgtggtggtgctgtcctttgagctgctgcacgctccagctaccgtgtgcggacccaagaagagcacaaacctggtgaagaataagggcagcggcggctctggctatatccccgaggctcctagagacggccaggcctacgtgcgcaaggatggcgagtgggtgctgctgtctaccttcctgggctctggctccggccagtacatcaaggccaactccaagtttatcggcatcacagagctgtga

SEQ ID NO: 55 - DNA, 864 bp, Artificial Sequence
  Codon-optimized nucleic acid sequence of RBD ext3-foldon-P2 of variant B.1.1.248 for CHO expression system
atgaagtgggtgaccttcatcagcctgctgttcctgttctcctccgcctattcccagcctaccgagagcatcgtgaggttccctaacatcacaaatctgtgcccattcggcgaggtgtttaacgccacccggtttgcctccgtgtacgcctggaacaggaagcggatcagcaattgcgtggctgactattctgtgctgtacaattccgcctccttctccaccttcaagtgctatggcgtgagcccaaccaagctgaacgacctgtgcttcacaaacgtgtacgctgactcttttgtgatcaggggcgatgaggtgcggcagatcgctccaggacagaccggcaccatcgctgactacaactataagctgcctgacgacttcaccggctgcgtgatcgcctggaactccaacaatctggattccaaagtgggcggcaactacaattatctgtacagactgttccgcaagtctaacctgaagccatttgagagagacatctccaccgaaatctaccaggctggcagcacaccatgcaacggagtgaagggcttcaattgttattttcccctgcagtcctacggcttccagcctacctacggcgtgggctatcagccataccgcgtggtggtgctgtcctttgagctgctgcacgctccagctaccgtgtgcggacccaagaagagcacaaacctggtgaagaataagggcagcggcggctctggctatatccccgaggctcctagagacggccaggcctacgtgcgcaaggatggcgagtgggtgctgctgtctaccttcctgggctctggctccggccagtacatcaaggccaactccaagtttatcggcatcacagagctgtga

SEQ ID NO: 56 - DNA, 864 bp, Artificial Sequence
  Codon-optimized nucleic acid sequence of RBD ext3-foldon-P2 of variant B.1.429 for CHO expression system
atgaagtgggtgaccttcatcagcctgctgttcctgttctcctccgcctattcccagcctaccgagagcatcgtgaggttccctaacatcacaaatctgtgcccattcggcgaggtgtttaacgccacccggtttgcctccgtgtacgcctggaacaggaagcggatcagcaattgcgtggctgactattctgtgctgtacaattccgcctccttctccaccttcaagtgctatggcgtgagcccaaccaagctgaacgacctgtgcttcacaaacgtgtacgctgactcttttgtgatcaggggcgatgaggtgcggcagatcgctccaggacagaccggcaagatcgctgactacaactataagctgcctgacgacttcaccggctgcgtgatcgcctggaactccaacaatctggattccaaagtgggcggcaactacaattatcggtacagactgttccgcaagtctaacctgaagccatttgagagagacatctccaccgaaatctaccaggctggcagcacaccatgcaacggagtggagggcttcaattgttattttcccctgcagtcctacggcttccagcctaccaatggcgtgggctatcagccataccgcgtggtggtgctgtcctttgagctgctgcacgctccagctaccgtgtgcggacccaagaagagcacaaacctggtgaagaataagggcagcggcggctctggctatatccccgaggctcctagagacggccaggcctacgtgcgcaaggatggcgagtgggtgctgctgtctaccttcctgggctctggctccggccagtacatcaaggccaactccaagtttatcggcatcacagagctgtga

SEQ ID NO: 57 - DNA, 792 bp, Artificial Sequence
  Codon-optimized nucleic acid sequence of RBD ext1-P2 of variant B.1.1.7 for insect expression system
atgaagtgggtgaccttcatcagcctgctgttcctgttctccagcgcctactcacagccaaccgagtccatcgtcaggttcccaaacatcactaacctgtgccctttcggtgaagtgttcaacgctaccagattcgcctccgtctacgcttggaaccgcaagcgtatctcaaactgcgtcgccgactactccgtgctgtacaactctgcttcattctccactttcaagtgctacggagtgtcacctaccaagctgaacgacctgtgcttcactaacgtctacgccgactccttcgtgatccgcggtgacgaggtccgtcagatcgctcctggacagaccggcaagatcgctgactacaactacaagctgccagacgacttcactggctgcgtgatcgcttggaacagcaacaacctggactctaaggtcggtggcaactacaactacctgtacaggctgttcagaaagtcaaacctgaagcctttcgagcgcgacatcagcaccgaaatctaccaggccggttctactccctgcaacggcgtggagggattcaactgctacttccccctgcagtcctacggcttccagccaacctacggcgtcggataccagccttaccgcgtggtcgtgctgagcttcgaactgctccacgctcctgctactgtctgcggacccaagaagtctactaacctggtcaagaacaagtgcgtgaacttcaacttcaacggaggtagcggttctggccagtacatcaaggctaactctaagttcatcggaatcactgaactgtaa

SEQ ID NO: 58 - DNA, 792 bp, Artificial Sequence
  Codon-optimized nucleic acid sequence of RBD ext1-P2 of variant B.1.351 for insect expression system
atgaagtgggtgaccttcatcagcctgctgttcctgttctccagcgcctactcacagccaaccgagtccatcgtcaggttcccaaacatcactaacctgtgccctttcggtgaagtgttcaacgctaccagattcgcctccgtctacgcttggaaccgcaagcgtatctcaaactgcgtcgccgactactccgtgctgtacaactctgcttcattctccactttcaagtgctacggagtgtcacctaccaagctgaacgacctgtgcttcactaacgtctacgccgactccttcgtgatccgcggtgacgaggtccgtcagatcgctcctggacagaccggtaacatcgctgactacaactacaagctgccagacgacttcactggctgcgtgatcgcttggaacagcaacaacctggactctaaggtcggtggcaactacaactacctgtacaggctgttcagaaagtcaaacctgaagcctttcgagcgcgacatcagcaccgaaatctaccaggccggttctactccctgcaacggcgtgaagggattcaactgctacttccccctgcagtcctacggcttccagccaacctacggcgtcggataccagccttaccgcgtggtcgtgctgagcttcgagctgctccacgctcctgctactgtctgcggacccaagaagtctactaacctggtcaagaacaagtgcgtgaacttcaacttcaacggaggtagcggttctggccagtacatcaaggctaactctaagttcatcggaatcactgaactgtaa

SEQ ID NO: 59 - DNA, 792 bp, Artificial Sequence
  Codon-optimized nucleic acid sequence of RBD ext1-P2 of variant B.1.1.248 for insect expression system
atgaagtgggtgaccttcatcagcctgctgttcctgttctccagcgcctactcacagccaaccgagtccatcgtcaggttcccaaacatcactaacctgtgccctttcggtgaagtgttcaacgctaccagattcgcctccgtctacgcttggaaccgcaagcgtatctcaaactgcgtcgccgactactccgtgctgtacaactctgcttcattctccactttcaagtgctacggagtgtcacctaccaagctgaacgacctgtgcttcactaacgtctacgccgactccttcgtgatccgcggtgacgaggtccgtcagatcgctcctggacagaccggtactatcgctgactacaactacaagctgccagacgacttcactggctgcgtgatcgcttggaacagcaacaacctggactctaaggtcggtggcaactacaactacctgtacaggctgttcagaaagtcaaacctgaagcctttcgagcgcgacatcagcaccgaaatctaccaggccggttctactccctgcaacggcgtgaagggattcaactgctacttccccctgcagtcctacggcttccagccaacctacggcgtcggataccagccttaccgcgtggtcgtgctgagcttcgagctgctccacgctcctgctactgtctgcggacccaagaagtctactaacctggtcaagaacaagtgcgtgaacttcaacttcaacggaggtagcggttctggccagtacatcaaggctaactctaagttcatcggaatcactgaactgtaa

SEQ ID NO: 60 - DNA, 792 bp, Artificial Sequence
  Codon-optimized nucleic acid sequence of RBD ext1-P2 of variant B.1.429 for insect expression system
atgaagtgggtcacgttcatttccctcctgttcctgttctcaagtgcttactcacaaccaaccgagtccatcgtccgtttccctaacatcaccaacctgtgccctttcggagaggtgttcaacgctactcgcttcgcctccgtctacgcttggaaccgcaagcgtatcagcaactgcgtcgccgactactctgtgctgtacaactccgcttccttctctaccttcaagtgctacggtgtgagccctaccaagctgaacgacctgtgcttcactaacgtctacgccgactctttcgtgatccgcggcgacgaagtccgtcagatcgctcctggtcagaccggcaagatcgctgactacaactacaagctgcctgacgacttcactggttgcgtgatcgcttggaactcaaacaacctggactccaaggtcggtggcaactacaactacaggtacagactgttcaggaagagcaacctgaagcccttcgagagagacatctcaaccgaaatctaccaggccggctccactccatgcaacggagtggagggtttcaactgctacttcccactgcagtcttacggattccagcctactaacggcgtcggataccagccctaccgcgtggtcgtgctgtcattcgaactgctccacgctcctgctactgtctgcggacccaagaagtccactaacctggtcaagaacaagtgcgtgaacttcaacttcaacggaggttctggcagcggacaatacatcaaggcaaacagcaaattcatcggcattacggaactctaa

SEQ ID NO: 61 - DNA, 864 bp, Artificial Sequence
  Codon-optimized nucleic acid sequence of RBD- ext3-foldon-P2 of variant B.1.1.7 for insect expression system
atgaagtgggtcactttcatcagcctgctgttcctgttctccagcgcttacagccagcctaccgaatcaatcgtccgtttcccaaacatcactaacctgtgccctttcggagaggtgttcaacgccacccgtttcgcttccgtgtacgcctggaacaggaagagaatcagcaactgcgtcgctgactactctgtgctgtacaactcagcctccttcagcaccttcaagtgctacggcgtgtcacccactaagctgaacgacctgtgcttcaccaacgtctacgccgactccttcgtgatcaggggagacgaggtcagacagatcgctccaggtcaaactggcaagatcgccgactacaactacaagctgcctgacgacttcaccggctgcgtcatcgcttggaacagcaacaacctggactctaaagtgggtggcaactacaactacctgtaccgcctgttccgtaagtcaaacctgaagcccttcgagcgcgacatctcaactgaaatctaccaggctggttccaccccatgcaacggagtcgagggtttcaactgctacttccctctgcaatcctacggtttccagcccacttacggagtgggttaccagccataccgtgtggtcgtgctgagcttcgaactgctgcacgcccctgctactgtgtgcggtcccaagaagagcaccaacctggtcaagaacaagggaagcggtggctccggttacatccctgaagctccccgcgacggacaggcctacgtccgtaaggacggagagtgggtgctgctgtcaactttcctgggatctggttcaggccagtacatcaaggctaactccaagttcatcggtatcaccgaactgtaa

SEQ ID NO: 62 - DNA, 864 bp, Artificial Sequence
  Codon-optimized nucleic acid sequence of RBD- ext3-foldon-P2 of variant B.1.351 for insect expression system
atgaagtgggtcactttcatcagcctgctgttcctgttctccagcgcttacagccagcctaccgaatcaatcgtccgtttcccaaacatcactaacctgtgccctttcggagaggtgttcaacgccacccgtttcgcttccgtgtacgcctggaacaggaagagaatcagcaactgcgtcgctgactactctgtgctgtacaactcagcctccttcagcaccttcaagtgctacggcgtgtcacccactaagctgaacgacctgtgcttcaccaacgtctacgccgactccttcgtgatcaggggagacgaggtcagacagatcgctccaggtcaaactggcaacatcgccgactacaactacaagctgcctgacgacttcaccggctgcgtcatcgcttggaacagcaacaacctggactctaaagtgggtggcaactacaactacctgtaccgcctgttccgtaagtcaaacctgaagcccttcgagcgcgacatctcaactgaaatctaccaggctggttccaccccatgcaacggagtcaagggtttcaactgctacttccctctgcaatcctacggtttccagcccacttacggagtgggttaccagccataccgtgtggtcgtgctgagcttcgaactgctgcacgcccctgctactgtgtgcggtcccaagaagagcaccaacctggtcaagaacaagggaagcggtggctccggttacatccctgaagctccccgcgacggacaggcctacgtccgtaaggacggagagtgggtgctgctgtcaactttcctgggatctggttcaggccagtacatcaaggctaactccaagttcatcggtatcaccgaactgtaa

SEQ ID NO: 63 - DNA, 864 bp, Artificial Sequence
  Codon-optimized nucleic acid sequence of RBD- ext3-foldon-P2 of variant B.1.1.248 for insect expression system
atgaagtgggtcactttcatcagcctgctgttcctgttctccagcgcttacagccagcctaccgaatcaatcgtccgtttcccaaacatcactaacctgtgccctttcggagaggtgttcaacgccacccgtttcgcttccgtgtacgcctggaacaggaagagaatcagcaactgcgtcgctgactactctgtgctgtacaactcagcctccttcagcaccttcaagtgctacggcgtgtcacccactaagctgaacgacctgtgcttcaccaacgtctacgccgactccttcgtgatcaggggagacgaggtcagacagatcgctccaggtcaaactggcacgatcgccgactacaactacaagctgcctgacgacttcaccggctgcgtcatcgcttggaacagcaacaacctggactctaaagtgggtggcaactacaactacctgtaccgcctgttccgtaagtcaaacctgaagcccttcgagcgcgacatctcaactgaaatctaccaggctggttccaccccatgcaacggagtcaagggtttcaactgctacttccctctgcaatcctacggtttccagcccacttacggagtgggttaccagccataccgtgtggtcgtgctgagcttcgaactgctgcacgcccctgctactgtgtgcggtcccaagaagagcaccaacctggtcaagaacaagggaagcggtggctccggttacatccctgaagctccccgcgacggacaggcctacgtccgtaaggacggagagtgggtgctgctgtcaactttcctgggatctggttcaggccagtacatcaaggctaactccaagttcatcggtatcaccgaactgtaa

SEQ ID NO: 64 - DNA, 864 bp, Artificial Sequence
  Codon-optimized nucleic acid sequence of RBD- ext3-foldon-P2 of variant B.1.429 for insect expression system
atgaagtgggtcactttcatcagcctgctgttcctgttctccagcgcttacagccagcctaccgaatcaatcgtccgtttcccaaacatcactaacctgtgccctttcggagaggtgttcaacgccacccgtttcgcttccgtgtacgcctggaacaggaagagaatcagcaactgcgtcgctgactactctgtgctgtacaactcagcctccttcagcaccttcaagtgctacggcgtgtcacccactaagctgaacgacctgtgcttcaccaacgtctacgccgactccttcgtgatcaggggagacgaggtcagacagatcgctccaggtcaaactggcaagatcgccgactacaactacaagctgcctgacgacttcaccggctgcgtcatcgcttggaacagcaacaacctggactctaaagtgggtggcaactacaactaccggtaccgcctgttccgtaagtcaaacctgaagcccttcgagcgcgacatctcaactgaaatctaccaggctggttccaccccatgcaacggagtcgagggtttcaactgctacttccctctgcaatcctacggtttccagcccactaacggagtgggttaccagccataccgtgtggtcgtgctgagcttcgaactgctgcacgcccctgctactgtgtgcggtcccaagaagagcaccaacctggtcaagaacaagggaagcggtggctccggttacatccctgaagctccccgcgacggacaggcctacgtccgtaaggacggagagtgggtgctgctgtcaactttcctgggatctggttcaggccagtacatcaaggctaactccaagttcatcggtatcaccgaactgtaa

SEQ ID NO: 65 - protein, 1204 aa, Artificial Sequence
  SK-S-trimer-P2  recombinant antigen
MKWVTFISLLFLFSSAYSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGSGGSGYIPEAPRDGQAYVRKDGEWVLLSTFLGSGSGQYIKANSKFIGITEL

SEQ ID NO: 66 - DNA, 3600 bp, Artificial Sequence
  Codon-optimized nucleic acid sequence of SK-S- trimer-P2 antigen for CHO expression system
atgttcgtgtttctggtgctgctgccactggtgtccagccagtgcgtgaacctgaccacaagaacccagctgccccctgcctataccaatagcttcacaaggggcgtgtactatcccgataaggtgttcaggtcctccgtgctgcacagcacacaggacctgtttctgcctttcttttctaacgtgacctggttccacgctatccacgtgtccggcaccaatggcacaaagaggttcgataatccagtgctgccctttaacgacggcgtgtacttcgcctccaccgagaagagcaacatcatccggggctggatctttggcaccacactggattctaagacacagtccctgctgatcgtgaacaatgctaccaacgtggtcatcaaggtgtgcgagttccagttttgtaatgacccattcctgggcgtgtactatcataagaacaataagagctggatggagtctgagtttcgcgtgtatagctctgccaacaattgtacatttgagtacgtgagccagcccttcctgatggatctggagggcaagcagggcaatttcaagaacctgagagagttcgtgtttaagaatatcgacggctacttcaaaatctactctaagcacaccccaatcaacctggtgcgcgatctgccacagggcttctccgccctggagccactggtggacctgcccatcggcatcaacatcaccaggtttcagacactgctggccctgcatcggtcttacctgacaccaggcgattccagctctggatggaccgctggcgccgctgcctactatgtgggctacctccagcccagaaccttcctgctgaagtacaacgagaatggcaccatcacagacgctgtggattgcgccctggaccccctgtctgagacaaagtgtacactgaagtcctttaccgtggagaagggcatctatcagacatccaatttcagagtgcagcctaccgagagcatcgtgcgctttcccaatatcacaaacctgtgcccttttggcgaggtgttcaacgctacccgcttcgcctccgtgtacgcttggaatagaaagcgcatcagcaactgcgtggccgattattctgtgctgtacaactccgcctccttctccaccttcaagtgctatggcgtgagccccacaaagctgaatgacctgtgctttaccaacgtgtacgctgattctttcgtgatcagaggcgacgaggtgcgccagatcgcccctggccagacaggcaagatcgctgattacaattataagctgcctgacgatttcaccggctgcgtgatcgcctggaacagcaacaatctggactctaaagtgggcggcaactacaattatctgtacaggctgtttcggaagtccaatctgaagccattcgagagagacatcagcacagaaatctaccaggctggctctaccccctgcaatggcgtggagggctttaactgttatttccctctccagagctacggcttccagccaaccaacggcgtgggctatcagccctaccgcgtggtggtgctgtcctttgagctgctgcacgctcctgctacagtgtgcggcccaaagaagagcaccaatctggtgaagaacaagtgcgtgaacttcaacttcaacggcctgaccggcacaggcgtgctgaccgagtccaacaagaagttcctgccttttcagcagttcggcagagacatcgccgataccacagacgctgtgcgcgatcctcagaccctggagatcctggacatcacaccatgctccttcggcggcgtgagcgtgatcacaccaggcaccaatacaagcaaccaggtggccgtgctgtatcaggatgtgaattgtaccgaggtgcccgtggctatccacgctgaccagctgacccctacatggagggtgtactctaccggctccaacgtgtttcagacacgggccggatgtctgatcggagctgagcatgtgaacaattcctatgagtgcgacatccctatcggcgccggcatctgtgcctcctaccagacccagacaaacagcccaaggcgggccaggtctgtggcttcccagagcatcatcgcctataccatgtccctgggcgccgagaatagcgtggcttacagcaacaattctatcgctatccctaccaacttcacaatctctgtgaccacagagatcctgccagtgtctatgaccaagacatccgtggattgcacaatgtatatctgtggcgactccaccgagtgcagcaacctgctgctccagtacggctccttttgtacccagctgaatagagccctgacaggcatcgctgtggagcaggacaagaacacacaggaggtgttcgcccaggtgaagcaaatctacaagaccccacccatcaaggattttggcggcttcaatttttcccagatcctgcccgacccttccaagcccagcaagaggtcttttatcgaggatctgctgttcaacaaggtgaccctggctgacgccggcttcatcaagcagtatggcgattgcctgggcgacatcgctgccagggacctgatctgcgcccagaagtttaatggcctgaccgtgctgcctccactgctgacagacgagatgatcgctcagtacacatctgctctgctggccggcaccatcacatccggatggaccttcggcgctggagccgccctccagatcccttttgccatgcagatggcttatcggttcaacggcatcggcgtgacccagaatgtgctgtacgagaaccagaagctgatcgccaatcagtttaactctgctatcggcaagatccaggattctctgtccagcacagcttccgccctgggcaagctccaggacgtggtgaatcagaacgctcaggccctgaataccctggtgaagcagctgtcctccaacttcggcgccatcagctctgtgctgaatgacatcctgtccaggctggacaaggtggaggctgaggtgcagatcgacaggctgatcaccggcaggctccagtccctccagacctacgtgacacagcagctgatcagagctgccgagatccgcgcttccgccaacctggctgccaccaagatgtccgagtgcgtgctgggacagagcaagagggtggatttttgtggcaagggctatcacctgatgtctttcccacagtccgcccctcacggcgtggtgtttctgcatgtgacctacgtgccagctcaggagaagaacttcaccacagctccagccatctgccacgacggcaaggctcattttcctagagagggcgtgttcgtgagcaacggcacccattggtttgtgacacagcgcaatttctatgagccacagatcatcaccacagataatacatttgtgagcggcaactgtgacgtggtcatcggcatcgtgaacaataccgtgtacgatcctctccagccagagctggactctggaagcggtggctccggctacatccccgaggccccccgcgacggccaggcctacgtgcgcaaggacggcgagtgggtgctgctgtccaccttcctgggaagcggtggctcccagtacatcaaggccaactccaagttcatcggcatcaccgagctgtaa

SEQ ID NO: 67 - DNA, 3615 bp, Artificial Sequence
  Codon-optimized nucleic acid sequence of SK-S- trimer-P2 antigen for BEV expression system
atgaagtgggtcactttcatcagcctgctgttcctgttctccagcgcttactctcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcaggtagcggaggtagcggatatattcctgaggctccccgcgacggacaggcttacgtccgcaaggatggtgaatgggtgctgctctccaccttcctcggcagcggaagcggacagtatatcaaggctaactccaagttcattggcatcaccgagttgtaa

(19) United States
(12) Patent Application Publication
(10) Pub. No.: US 2023/0257425 A1
(43) Pub. Date: Aug. 17, 2023

(54) VACCINE COMPOSITION FOR PREVENTING OR TREATING INFECTION OF SARS-COV-2

(71) Applicant: SK BIOSCIENCE CO., LTD., Gyeonggi-do (KR)

(72) Inventors: Teawoo KWON, Gyeonggi-do (KR); YoonJae LEE, Gyeonggi-do (KR); Eun-som KIM, Gyeonggi-do (KR); Seung-hye HONG, Gyeonggi-do (KR); Ki-weon SEO, Gyeonggi-do (KR); Sujeen LEE, Gyeonggi-do (KR)

(21) Appl. No.: 17/922,407

(22) PCT Filed: Apr. 29, 2021
(86) PCT No.: PCT/KR2021/005488

§ 371 Date: Oct. 31, 2022

(30) Foreign Application Priority Data
Apr. 29, 2020 (KR) 10-2020-0052855
Sep. 9, 2020 (KR) 10-2020-0115694
Sep. 23, 2020 (KR) 10-2020-0123308
Dec. 1, 2020 (KR) 10-2020-0166091

(51) Int. Cl. C07K 14/165; C07K 14/33; C12N 15/62; A61P 31/14

(52) U.S. Cl. CPC C07K 14/165; C07K 14/33; C12N 15/62; A61P 31/14; A61K 2039/55505

(57) ABSTRACT

Provided is a recombinant protein for preventing or treating infection of SARS-Coronavirus-2 antigen comprising an extended receptor binding domain (RBD) of a spike protein of SARS-Coronavirus-2, and a vaccine composition comprising thereof. Also the present invention relates to a method for preventing infection of SARS-Coronavirus-2 by administering the recombinant antigen protein to a subject. The present invention can prevent COVID-19 infection. The present invention can be used as a vaccine.

### CROSS-REFERENCE TO RELATED APPLICATIONS

This application is a national phase application of PCT Application No. PCT/KR2021/005488, filed on Apr. 29, 2021, which claims benefit to Korean Patent Application Nos. 10-2020-0052855, filed on Apr. 29, 2020, 10-2020-0115694, filed on Sep. 9, 2020, 10-2020-0123308, filed on Sep. 23, 2020 and 10-2020-0166091, filed on Dec. 1, 2020. The entire disclosure of the applications identified in this paragraph are incorporated herein by reference.

### TECHNICAL FIELD

The present application claims priority to Korean Patent Application No. 10-2020-0166091 filed on Dec. 1, 2020, Korean Patent Application No. 10-2020-0123308 filed on Sep. 23, 2020, Korean Patent Application No. 10-2020-0115694 filed on Sep. 9, 2020, and Korean Patent Application No. 10-2020-0052855 filed on Apr. 29, 2020 in the Republic of Korea, and all contents disclosed in the specification and drawings of the applications are incorporated herein by reference. The present invention relates to a vaccine composition for preventing or treating infection of SARS-Coronavirus-2 (SARS-CoV-2). More specifically, it relates to a vaccine composition for preventing or treating infection of SARS-Coronavirus-2 using a recombinant protein.

### BACKGROUND ART

SARS-Coronavirus-2 (SARS-CoV-2) is called Severe Acute Respiratory Syndrome Coronavirus 2 or COVID19, and in South Korea it is named Corona 19. SARS-Coronavirus-2 is a virus first discovered at Huanan Fish Market in Wuhan on Dec. 12, 2019. It is an RNA virus, and a Human-to-human infection has been confirmed.

SARS-Coronavirus-2 is a virus that needs to be handled in a biosafety level 3 research facility (BSL3 facility), and its reproduction index (RO) is estimated to be 1.4 to 3.9. This means that one patient can transmit the virus to a minimum of 1.4 persons and a maximum of 3.9 persons. In other words, it is estimated that the control of infectious diseases by SARS-Coronavirus-2 is quite difficult, and as of Mar. 31, 2020, 785,867 infected and 37,827 deaths worldwide were counted.

Symptoms such as fever, shortness of breath, kidney and liver damage, cough, and pneumonia are observed for 2 to 14 days after infection with the virus, and treatments have not yet been developed.

In a situation where no treatment has been developed, research on vaccines is urgently needed to prevent infection and prevent spread to the community. Because the pandemic virus is usually a high-risk pathogen, in the case of an inactivated vaccine and a live vaccine, there is a high risk in the production and human administration of vaccine substances. In particular, in the case of a live vaccine, it takes a very long time to attenuate and prove its safety. The present inventors have completed the present invention by studying a recombinant protein vaccine applicable to a new infectious disease in the current pandemic in terms of versatility, safety, efficacy and commercialization.
1. Zhou Z, Post P, Chubet R, et al. A recombinant baculovirus-expressed S glycoprotein vaccine elicits high titers of SARS-associated coronavirus (SARS-CoV) neutralizing antibodies in mice. Vaccine. 2006; 24(17):3624-3631.
2. Dai L, Zheng T, Xu K, et al. A Universal Design of Betacoronavirus Vaccines against COVID-19, MERS, and SARS. Cell. 2020; 182(3):722-733.e11.

### DISCLOSURE

### Technical Problem

Accordingly, in order to solve the above problems, the present invention is to provide a novel recombinant protein antigen for preventing or treating infection of SARS-Coronavirus-2, a vaccine composition comprising the antigen or a method for preparing thereof. The present invention is to provide a recombinant protein vaccine, a method for preventing or treating infection of SARS-Coronavirus-2 using thereof or a use of the recombinant protein vaccine for preventing or treating infection of SARS-Coronavirus-2. The present invention is to provide a novel recombinant protein for preventing or treating infection of SARS-Coronavirus-2 (SARS-CoV-2) that can be expected to reduce the amount of virus in the body by not only producing a neutralizing antibody but also fighting off virus that infected cells.

### Technical Solution

In one aspect of the present invention, the present invention provides a recombinant protein for preventing or treating infection of SARS-Coronavirus-2 (SARS-CoV-2), a gene construct for expressing the antigen protein, or a vaccine composition comprising the recombinant protein.

The present invention provides a recombinant protein for preventing or treating infection of SARS-Coronavirus-2 comprising an extended receptor binding domain (RBD) of a spike protein (S protein) of SARS-Coronavirus-2. Hereinafter, the receptor binding domain of the spike protein (S protein) of wild type SARS-Coronavirus-2 is referred to as ‘Covid-19 S RBP’, and the extended receptor binding domain of the spike protein of SARS-Coronavirus-2 of the present invention is referred to as ‘Extended_S_RBD’. The Extended_S_RBD polypeptide sequence may be preferably represented by SEQ ID NOs: 1, 6, 7, and 8. It may include all polypeptides having sequence homology of at least 70%, at least 80%, at least 90%, and at least 95% of the sequence.

SARS-CoV-2 is known to strongly adhere to the surface of a host cell through ACE2 (Angiotensin Converting Enzyme2) receptor, and the RBD (Receptor-Binding Domain) of the spike protein of SARS-CoV-2 is known to be used to bind to the ACE2 receptor. The RBD contained in the spike protein of SARS-CoV-2 used in the RBD crystal structure in one embodiment of the present invention has a polypeptide located at 331 to 524 of the full-length polypeptide sequence of the spike protein, which is represented by SEQ ID NO: 37.

The present inventors completed the present invention by confirming that when including the RBD region of the spike protein of SARS-CoV-2 and further including a polypeptide sequence at the C-terminus and N-terminus, structural stability of an antigen protein, formation of a stable disulfide bond, increase in consistency of glycosylation pattern, increase in antigen size, increase in immunogenicity, increase in consistency of disulfide bond pattern, etc., which are difficult to achieve with the RBD region of the spike protein alone, are achieved. Further, the present inventors did not know exactly the specific reason, but it was confirmed that the recombinant protein of the present invention has excellent cell-mediated immunity inducing effect, and a high neutralizing antibody titer.

The term “extended receptor binding domain of a spike protein of SARS-Coronavirus-2 (Extended_S_RBD)” used herein refers to a form in which at least 5 polypeptide sequences are further included in the C-terminal and N-terminal directions of the domain while including a polypeptide that forms the receptor binding domain of the spike protein of SARS-CoV-2 (polypeptide sequence corresponding to positions 331 to 524 of the S protein, a polypeptide of SEQ ID NO: 33). Specifically, it includes the polypeptide of SEQ ID NO: 33, and has a form in which 5, 6, 7, 8, 9, 10, 11, 12, 13, 14, 15, 16, 17, 18, 19, 20, 21, 22, 23, 24, 25, 26, 27, 28, 29, 30 or more of the polypeptide sequences of the S protein are extended respectively in the N-terminal and C-terminal directions of the polypeptide. Furthermore, the Extended_S_RBD may include a polypeptide corresponding to positions 14 to 1214 based on FIG. 1. More specifically, at least 5 to 25 optional polypeptide sequences may be further extended in the C-terminal and N-terminal directions of the wild type RBD polypeptide sequence of SEQ ID NO: 33. Preferably, the Extended_S_RBD may have a polypeptide sequence corresponding to positions 328 to 531 (SEQ ID NO: 1), 321 to 545 (SEQ ID NO: 6), 321 to 591 (SEQ ID NO: 7), and/or 321 to 537 (SEQ ID NO: 8) of the polypeptide sequence of the spike protein. In particular, a recombinant protein comprising a polypeptide sequence corresponding to positions 321 to 545 (SEQ ID NO: 6), 321 to 591 (SEQ ID NO: 7), and/or 321 to 537 (SEQ ID NO: 8), or a polypeptide comprising or consisting of peptide sequences that are at least 75%, 80%, 85%, 90%, 91%, 92%, 93%, 94%, 95%, 96%, 97%, 98%, 99% or more, or 100% identical to the sequence can express glycosylated antigen in a single pattern in the virus expression system of the present invention. In particular, it can express the glycosylated antigen in a single pattern in the baculovirus expression system. Further, an antigen protein including the Extended_S_RBD according to one embodiment of the present invention can eliminate unwanted disulfide bonds and increase the consistency of disulfide bond patterns, making it easy to control protein refolding and maintain the three-dimensional structure of the protein stably. In addition, a construct expressing a protein having the above polypeptide sequence can increase protein production. Further, the recombinant protein of the present invention including the Extended_S_RBD is excellent in increasing the immune-inducing response.

The term “recombinant protein” used herein refers to a protein that can function as an antigen that can be used for preventing or treating infection of SARS-CoV-2, and specifically, contains a polypeptide sequence of a certain section, selected at a certain position of the spike protein of SARS-CoV-2. The recombinant protein refers to a protein artificially made through cleavage of a partial region of the spike protein of SARS-CoV-2, and binding to a foreign gene. The recombinant protein may include a functional fragment or analog of the recombinant protein. The functional fragment or analog may be included in the scope of the present invention if it has functional identity even if a part of the polypeptide sequence of the recombinant protein is deleted, added, or substituted. Deletion, addition, or substitution of a part of the sequence may include deletion, addition, or substitution of at least 1, 2, 3, 4, 5, 6, or more polypeptides. The fragment and/or analog may comprise or consist of peptide sequences that are at least 75%, 80%, 85%, 90%, 91%, 92%, 93%, 94%, 95%, 96%, 97%, 98%, 99% or more, or 100% identical to the recombinant protein, and may have functional identity. The meaning of having the functional identity means that the recombinant protein limited to the sequence herein can achieve the desired effect.

In one aspect, the Extended_S_RBD may optionally further include a T cell epitope at the C-terminus and/or N-terminus, and preferably may further include a T cell epitope at the C-terminus. The T cell epitope may be used without limitation as long as it is a T cell epitope domain used to manufacture a vaccine, and preferably, it may comprise or consist of the polypeptide sequence of one of the T cell epitope, Tetanus Toxoid Epitope P2 domain (SEQ ID NO: 3) or peptide sequences that are at least 75%, 80%, 85%, 90%, 91%, 92%, 93%, 94%, 95%, 96%, 97%, 98%, 99% or more, or 100% identical to the above sequence. By binding the P2 domain to the recombinant protein, it may exhibit an improved immune enhancing effect. In another embodiment, the extended receptor binding domain (RBD) may be linked to the foldon domain, and the foldon domain may provide a recombinant protein linked to the P2 domain. The foldon domain may have any foldon sequence known to those skilled in the art. Preferably, it may include a foldon of bacteriophage T4 fibritin, and may include a polypeptide comprising or consisting of the sequence of SEQ ID NO: 4 or peptide sequences that are at least 75%, 80%, 85%, 90%, 91%, 92%, 93%, 94%, 95%, 96%, 97%, 98%, 99% or more, or 100% identical to the above sequence. The foldon domain can induce an antigen to form a trimer, thereby increasing the size of the antigen and increasing antigenicity.

The P2 peptide and/or foldon peptide may be provided in a form linked to the Extended_S_RBD through a linker. The linkage may be linked by a linker consisting of at least three polypeptides. For example, the linker is 16 polypeptides or less in length and may preferably consist of 6 or less polypeptides. The polypeptides used in the linker may be at least one of G (Gly, glycine), S (Ser, serine), and A (Ala, alanine). Preferably, the linker may be at least one peptide linker selected from the group consisting of Gly-Ser-Gly-Ser-Gly (GSGSG), Gly-Ser-Ser-Gly (GSSG), Gly-Ser-Gly-Gly-Ser (GSGGS), Gly-Ser-Gly-Ser (GSGS), and Gly-Ser-Gly-Ser-Ser-Gly (GSGSSG), and preferably may be a GSGSG peptide linker for the purpose of the present invention. The foldon domain and the P2 domain may also be linked with the same linker or different linker, and preferably may be linked with the same linker. Preferably, the linkage may be linked with a GSGSG peptide linker for the purpose of the present invention. One embodiment of the present invention provides at least one recombinant protein selected from SEQ ID NOs: 1, 6 to 13 and 44 to 48 and SEQ ID NO: 65, or a recombinant protein comprising or consisting of peptide sequences that are at least 75%, 80%, 85%, 90%, 91%, 92%, 93%, 94%, 95%, 96%, 97%, 98%, 99% or more, or 100% identical to the above sequence. Preferably, it provides at least one recombinant protein selected from SEQ ID NOs: 1 and 6 to 13, and preferably it includes at least one recombinant protein selected from SEQ ID NOs: 9 to 13. The recombinant protein has excellent reactivity with an antibody, can provide high neutralizing antibody titer, and induces excellent cell-mediated immunity reaction. Further, when the substance immunized with the vaccine of the present invention (or recombinant protein antigen) is memorized in T cells, the cells secret IFN, a cytokine by a stimulating antigen, and can activate immunity. While the existing vaccine is only aimed at preventing infection by using a neutralizing antibody, the present invention can contribute to suppression of transmissibility after infection. The vaccine of the present invention can have excellent effects on activating T cells and destroying virus infected by the activated T cells.

One embodiment of the present invention can provide a gene construct for producing a recombinant protein for preventing or treating infection of SARS-Coronavirus-2 antigen. The term “gene construct” used herein is understood to mean the smallest element for protein expression in a cell or a nucleic acid molecule containing only the smallest element. The gene construct can be provided as an antigen expression construct for expressing a recombinant protein antigen. The gene construct for producing a recombinant protein for preventing or treating infection of SARS-Coronavirus-2 antigen may include an open reading frame containing a polynucleotide sequence encoding the Extended_S_RBD. For example, in order to express at least one recombinant protein antigen selected from the group consisting of SEQ ID NOs: 1, 6 to 13, 44 to 48, and SEQ ID NO: 65, a codon-optimized gene construct can be provided. The gene construct may be sequentially linked to the open reading frame so that the polynucleotide encoding the heterologous signal peptide is operable. When a base sequence is arranged in a functional relationship with another nucleic acid sequence, it is “operably linked”. These can be genes or regulatory sequences linked in a way that allows gene expression when an appropriate molecule (e.g., transcription activating protein) is bound to the regulatory sequences. Polypeptide encoding the heterologous signal peptide can be added to increase the amount of protein secretion and increase the yield of antigen production.

By linking the polynucleotide encoding the P2 domain of Tetanus toxin, the gene construct may provide a nucleotide in which the polynucleotides encoding the heterologous signal peptide, the open reading frame, and the P2 domain of Tetanus toxin, respectively, are linked (more specifically, operably linked). The gene construct can provide a codon-optimized polynucleotide by further linking the polynucleotide encoding the foldon domain between the extended receptor binding domain and the P2 domain of Tetanus toxin. The linkage may be linked by a polynucleotide encoding a linker consisting of at least three polypeptides. The gene construct may include at least one polynucleotide selected from the group consisting of SEQ ID NOs: 14 to 25 or SEQ ID NOs: 49 to 64, or a polynucleotide comprising or consisting of sequences that are at least 75%, 80%, 85%, 90%, 91%, 92%, 93%, 94%, 95%, 96%, 97%, 98%, 99% or more, or 100% identical thereto. Preferably, one embodiment of the present invention provides a codon-optimized nucleotide sequence to obtain an excellent recombinant protein in the baculovirus expression system. It may include at least one nucleotide sequence selected from the group consisting of polynucleotide sequence of SEQ ID NO: 14 (SK-RBD), polynucleotide sequence of SEQ ID NO: 16 (SK-RBD-P2), polynucleotide sequence of SEQ ID NO: 18 (SK-RBD-EX1-P2), polynucleotide sequence of SEQ ID NO: 20 (SK-RBD-EX2-P2), polynucleotide sequence of SEQ ID NO: 22 (SK-RBD-EX3-P2), and polynucleotide sequence of SEQ ID NO: 24 (SK-RBD-Foldon-P2). Or, preferably, one embodiment of the present invention provides a codon-optimized nucleotide sequence to obtain an excellent recombinant protein in the expression system using Chinese Hamster Ovary (CHO) cells as host cells. For example, it may include at least one polynucleotide sequence selected from the group consisting of polynucleotide sequence of SEQ ID NO: 15 (SK-RBD), polynucleotide sequence of SEQ ID NO: 17 (SK-RBD-P2), polynucleotide sequence of SEQ ID NO: 19 (SK-RBD-EX1-P2), polynucleotide sequence of SEQ ID NO: 21 (SK-RBD-EX2-P2), polynucleotide sequence of SEQ ID NO: 23 (SK-RBD-EX3-P2), and polynucleotide sequence of SEQ ID NO: 25 (SK-RBD-Foldon-P2). Preferably, the polynucleotide sequence is a DNA sequence.

The term “signal peptide” or “signal sequence” used herein is used interchangeably herein and refers to a short peptide present at the N-terminus of the newly synthesized polypeptide chain (Generally, it has a length of 5 to 30 polypeptides, but is not limited thereto) that directs the protein to the secretory pathway in the host cell. The signal peptide referred to herein is removed during protein secretion. The ‘heterologous signal peptide or signal sequence’ refers to a signal sequence introduced from outside or newly synthesized, not the signal sequence of the spike protein of SARS-CoV-2. Preferred heterologous signal sequence includes murine phosphatase signal peptide sequence, honeybee melittin signal peptide sequence, human albumin signal peptide sequence and the like, and preferably, for the purpose of the present invention, a human albumin signal peptide represented by SEQ ID NO: 2 can be used.

In one embodiment of the present invention, a recombinant expression vector comprising the gene construct is provided. The recombinant protein of the present invention can be prepared by cloning and expression in a prokaryotic or eukaryotic expression system using a suitable expression vector. Any method known in the art can be used. Preferably, in consideration of the purpose of the present invention and the protein expression rate, BEVS, CHO or E. coli expression system can be used, and preferably BEVS and/or CHO expression system can be used. The vector may be of any suitable type and may include, but is not limited to, phage, virus, plasmid, phagemid, cosmid, bacmid and the like. For example, a DNA molecule encoding the antigen of the present invention is inserted into an expression vector suitably prepared by a technique well known in the art. The known technique can be referred to Zhou Z, Post P, Chubet R, et al. A recombinant baculovirus-expressed S glycoprotein vaccine elicits high titers of SARS-associated coronavirus (SARS-CoV) neutralizing antibodies in mice. Vaccine. 2006; 24(17):3624-3631. doi:10.1016/j.vaccine.2006.01.059 (baculo system), Dai L, Zheng T, Xu K, et al. A Universal Design of Betacoronavirus Vaccines against COVID-19, MERS, and SARS. Cell. 2020; 182(3):722-733.e11. doi:10.1016/j.cell.2020.06.035 (CHO system) and the like.

The gene construct according to one embodiment of the present invention uses a baculovirus expression system (BEVS).

As the baculovirus expression system, a system already widely used for the production of a recombinant protein in the art can be used without limitation. For example, a commercially available baculovirus vector such as pBAC4x-1 (Novagen) can be used. Suitable baculovirus promoters used in the present invention are well known in the literature. As the baculovirus promoter, a commonly used promoter such as polyhedron and p10 promoter may be used. A recombinant bacmid obtained by transforming a baculovirus vector containing a gene construct containing a polynucleotide sequence encoding the antigen protein into E. coli, and a recombinant baculovirus containing the same as a genome are also provided. A host cell containing the recombinant bacmid or transfected with the recombinant baculovirus is also included in the scope of the present invention.

DNA molecules containing a polynucleotide sequence encoding the antigen protein of the present invention can be inserted into a vector having a transcription and translation control signal. A cell stably transformed by the introduced DNA can be selected by introducing one or more markers that allow selection of a host cell containing the expression vector. The marker may provide, for example, antibiotic resistance, deficient nutrient synthesis genes and the like. Once the vector or DNA sequence containing the construct has been prepared for expression, the DNA construct can be introduced into a suitable host cell by any one of various suitable means, that is, transformation, transfection, conjugation, protoplast fusion, electrophoration, calcium phosphate-precipitation, direct microinjection and the like.

The preferred host cell is a eukaryotic host cell, for example, and it may include Spodopterafrugiperda (Sf) cells such as Sf9 and Sf21 using the Baculovirus expression system as insect cells, Trichoplusiani cells such as Hi-5 cells, and Drosophila S2 cells, and may include Chinese Hamster Ovary (CHO) cells as mammalian cells. A suitable host cell line may be any Chinese Hamster Ovary (CHO) cell line. The term ‘host cell’ refers to a cell capable of growing in a culture solution and expressing the desired protein recombinant product. A suitable cell line may include, for example, CHO K1, CHO pro3-, CHO DG44, CHO P12 and the like, but not limited thereto.

A recombinant protein with excellent expression rate can be obtained through the host cell. As a non-limiting example, the eukaryotic host cell may include, for example, yeast, algae, plants, Caenorhabditis elegans (nematodes) and the like, and the prokaryotic cell may include, for example, bacterial cells such as E. coli, B. subtilis, Salmonella typhi, and mycobacteria within a range that does not interfere with the object of the present invention. After introduction of the vector, the host cell is grown in a general medium or a selection medium (selected for growth of a vector-containing cell). The desired protein is produced as a result of the expression of the cloned gene sequence(s). Purification of the recombinant protein may be performed by any known methods for the above purpose, that is, any conventional procedure involving extraction, precipitation, chromatography, electrophoresis and the like.

Another embodiment of the present invention provides a method for preparing the recombinant protein, and the method may comprises a step of culturing the host cell transformed with the vector containing the polynucleotide sequence of the present invention and isolating the desired product.

Another embodiment of the present invention provides a novel used of the recombinant protein antigen for preventing or treating infection of SARS-Coronavirus-2, and a SARS-Coronavirus-2 infection prevention method for preventing or treating infection of SARS-Coronavirus-2 by administering the antigen to a subject.

Another embodiment of the present invention provides a vaccine composition for preventing or treating infection of SARS-Coronavirus-2, which comprises the recombinant protein containing a polypeptide that forms the extended receptor binding domain (RBD) of the spike protein of SARS-Coronavirus-2 and a pharmaceutically acceptable carrier or excipient.

The term ‘SARS-Coronavirus-2 infection’ can be understood as a concept that broadly includes not only infection of SARS-Coronavirus-2 itself, but also various conditions (e.g., respiratory disease, pneumonia and the like) caused by infection of the virus. In the present invention, the vaccine can be prepared by a conventional method well known in the art, and may optionally further include several additives that can be used in the manufacture of a vaccine in the art. The vaccine composition according to the present invention may contain the recombinant protein antigen and a pharmaceutically acceptable carrier. For example, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate and mineral oil and the like that is commonly used in formulation may be included, but not limited thereto. In addition to the above ingredients, the pharmaceutical composition of the present invention further comprise non-ionic surfactants such as TWEEN™, polyethylene glycol (PEG), antioxidants including ascorbic acid, lubricants, wetting agents, sweetening agents, flavoring agents, emulsifying agents, suspending agents, preservatives and the like. In the present invention, the vaccine can be prepared in unit dosage form or be prepared by incorporating it into a multi-dose container by formulating using a pharmaceutically acceptable carrier and/or excipient according to a method that can be easily carried out by a person skilled in the art. In this case, the formulation may be in the form of a solution, suspension or emulsion in an oil or aqueous medium, or may be in the form of an extract, powder, granule, tablet or capsule. It may additionally include a dispersant or stabilizer. In the present invention, a suitable dosage of the vaccine may be prescribed in various ways depending on factors such as formulation method, mode of administration, patient's age, weight, sex and pathological condition, food, administration time, administration route, excretion rate and response sensitivity. On the other hand, the dosage of the vaccine according to the present invention may be preferably 1 to 500 ug per dose. In one embodiment of the present invention, the vaccine containing the recombinant protein as an active ingredient may be administered into the body by intravenous injection, intramuscular injection, subcutaneous injection, transdermal delivery, or airway inhalation, but is not limited thereto.

The vaccine composition may further include an immunological adjuvant to enhance the immune response effect, and may further include the nucleocapsid (N) protein of SARS-Coronavirus-2 with or without the immunological adjuvant.

For example, the immunological adjuvant may be at least one selected from AS03, CpG, squalene (MF59), liposome, TLR agonist, MPL (monophosphoryl lipid A) (AS04), magnesium hydroxide, magnesium carbonate hydroxide pentahydrate, titanium dioxide, calcium carbonate, barium oxide, barium hydroxide, barium peroxide, barium sulfate, calcium sulfate, calcium pyrophosphate, magnesium carbonate, magnesium oxide, aluminum hydroxide, aluminum phosphate and hydrated aluminum potassium sulfate (Alum), which is well known in the vaccine manufacturing industry, and preferably, it may include CpG, aluminum hydroxide, or a mixture thereof. Most preferably, it may include a mixture of CpG and aluminum hydroxide that has excellent immune induction effect and can induce high neutralizing antibody titer, but not limited thereto.

The ‘nucleocapsid (N) protein of SARS-Coronavirus-2’ includes the artificially made nucleocapsid (N) protein of SARS-Coronavirus-2 of SEQ ID NO: 26, and may include a fragment, and/or analog having functional identity thereto. The functional fragment or analog may be included in the scope of the present invention if it has functional identity even if a part of the polypeptide sequence of the protein of SEQ ID NO: 26 is deleted, added, or substituted. Deletion, addition, or substitution of a part of the sequence may include deletion, addition, or substitution of at least 1, 2, 3, 4, 5, 6, or more polypeptides. For example, deletion, substitution, or addition of any one or more of the residues of the polypeptide sequence of SEQ ID NO: 26 may be included, and for example, deletion of a residue at position 1 or at least one of the remaining residues of SEQ ID NO: 26 may be included. The fragment and/or analog may be at least 75%, 80%, 85%, 90%, 91%, 92%, 93%, 94%, 95%, 96%, 97%, 98%, 99%, or 100% identical to the sequence of SEQ ID NO: 26, and may have functional identity. The meaning of having the functional identity means that the N protein can achieve the purpose and effect similar to those desired in the present invention.

The N protein can induce cell-mediated immunity, and can induce increased protective immunogenicity by using it with the recombinant antigen protein obtained according to one embodiment of the present invention. The N protein has high stability and shows significant immunogenicity inducing ability, and cell-mediated immunity using it can effectively protect virus in the early stage of infection. Further, administration of the N protein may result in a high increase in a RBD-specific IgG titer. By the simultaneous administration of the recombinant protein antigen and N protein obtained according to one embodiment, improved cellular immunogenicity can be expected. In particular, it was confirmed that the simultaneous administration of the N protein can effectively protect the virus in the early stage of infection. The N protein is related to the induction of cytotoxic T lymphocytes, and may be used to induce cell-mediated immunity response of the vaccine obtained according to one embodiment.

The construct for N protein expression of the protein of SEQ ID NO: 26 may be provided by linking a polynucleotide sequence capable of expressing a human albumin signal peptide to the N-terminus of the N protein. Preferably, the polynucleotide sequence optimized in the BEV expression system is represented by SEQ ID NO: 28, and the polynucleotide sequence optimized in the CHO expression system is represented by SEQ ID NO: 29. A polynucleotide comprising or consisting of a nucleotide sequence that is at least 75%, 80%, 85%, 90%, 91%, 92%, 93%, 94%, 95%, 96%, 97%, 98%, 99% or more, or 100% identical to the sequence may also be included in the scope of the present invention. Optionally, the vaccine composition may further include a polypeptide constituting any one SARS-Coronavirus-2 derived protein selected from the group consisting of matrix (M) protein and small envelope (E) protein of SARS-Coronavirus-2. The vaccine composition preferably contains polypeptides constituting the recombinant protein and N protein, and may include the N protein and the recombinant protein in a mixing ratio (N protein: recombinant protein) of weight ratio of 1:1 to 500, preferably 1:1 to 400, preferably 1:1 to 300, preferably 1:1 to 200, preferably 1:1 to 100, preferably 1:1 to 80, preferably 1:30 to 50. When included in the above ratio, the binding force with the antibody is excellent, or a high neutralizing antibody titer can be confirmed.

Another embodiment of the present invention provides a method for evaluating an immune response in an animal, comprising a step of administering the recombinant protein antigen of the present invention, or (or specifically) at least one recombinant protein selected from the group consisting of SEQ ID NOs: 1, 6 to 13 and 44 to 48, and SEQ ID NO: 65, or a recombinant protein comprising or consisting of a peptide that is at least 75%, 80%, 85%, 90%, 91%, 92%, 93%, 94%, 95%, 96%, 97%, 98%, 99%, or 100% identical to the sequence to an animal. The method of evaluation the immune response may include the case of excluding humans. The method may evaluate the immune response by measuring a titer or a neutralizing antibody titer from an animal serum, and the IgG antibody titer may include an RBD-specific antibody titer, and/or an N protein-specific antibody titer. Herein, the term “animal” is not particularly limited, but may include animals including humans, dogs, cats, horses, sheep, pigs, cattle, poultry and fish, but may exclude humans.

One embodiment provides a method of increasing the specificity for an antibody by administering a composition comprising any one recombinant protein selected from the group consisting of SEQ ID NO: 1, SEQ ID NOs: 6 to 13, SEQ ID NOs: 44 to 48, and SEQ ID NO: 65 or a recombinant protein comprising or consisting of a peptide that is at least 75%, 80%, 85%, 90%, 91%, 92%, 93%, 94%, 95%, 96%, 97%, 98%, 99%, or 100% identical thereto to an animal, and comparing thereof to administering the peptide of Covid-19_S_RBP of SEQ ID NO: 37 or the S protein of SEQ ID NO: 34. The antibody may be an antibody contained in the serum isolated from humans. The composition may include the N protein of SEQ ID NO: 26 or a protein comprising or consisting of a peptide sequence that is at least 75%, 80%, 85%, 90%, 91%, 92%, 93%, 94%, 95%, 96%, 97%, 98%, 99%, or 100% identical thereto an at least one immunological adjuvant selected from the group consisting of aluminum hydroxide, CpG oligopolynucleotide and a mixture thereof.

### Advantageous Effects

The recombinant protein and/or recombinant virus vaccine according to one embodiment of the present invention has high safety.

The vaccine according to one embodiment of the present invention has excellent immunogenicity, and has excellent efficacy as a vaccine.

The vaccine of the present invention has high neutralizing antibody titer.

The vaccine of the present invention is excellent in the induction of cell-mediated immunity. While the existing vaccine aims only at preventing infection by using a neutralizing antibody, the present invention can contribute to suppression of propagation power after infection. The vaccine of the present invention can have an excellent effect on activating T cells and destroying the virus infected by the activated T cells.

The present invention has excellent preventative and therapeutic effects against SARS-Coronavirus-2 infection.

The recombinant protein of the present invention can maintain a stable three-dimensional RBD protein structure. It can have high a high antibody production rate by using the recombinant antigen of the present invention.

A synthetic antigen vaccine consisting of the RBD protein, which is a major antigen, has an advantage of minimizing side effects such as Antibody-dependent effect (ADE), which induces large amounts of antibodies without neutralizing ability.

The vaccine of the present invention can be stored at a refrigerated temperature of 2 to 8° C. Therefore, it has advantages of easier distribution, fewer side effects, and safety.

### DESCRIPTION OF DRAWINGS

The accompanying drawings illustrate a preferred embodiment of the present invention and together with the foregoing invention, serve to provide further understanding of the technical features of the present invention, and thus, the present invention is not construed as being limited to the drawing.

FIG. 1 is a schematic diagram showing a structure of SARS-CoV2 spike full-length protein domain.

FIG. 2 shows a schematic diagram of a construct for expression of a recombinant protein antigen (SK-RBD, SK-RBD-P2, SK-RBD-Ex1-P2, SK-RBD-Ex2-P2, SK-RBD-Ex3-P2) prepared based on the peptide sequence of an S protein. For example, in the case of the gene construct called SK-RBD, SP 1˜18 shows the form in which the open reading frame of the polynucleotide encoding the signal peptide having 18 polypeptide sequences is operably linked to the open reading frame of the polynucleotide encoding the peptide sequence of SK-RBD. In the case of the gene construct called SK-RBD-P2, SP 1˜18 shows the form in which the open reading frame of the polynucleotide encoding the signal peptide having 18 polypeptide sequences is operably linked to the open reading frame of the polynucleotide encoding the peptide sequence of SK-RBD, and the polynucleotide encoding the P2 domain is linked thereto.

FIG. 3 is an electrophoresis picture showing that the recombinant antigen prepared in one embodiment of the present invention forms a stable three-dimensional structure.

FIGS. 4A and 4B show the results of weight comparison and survival.

FIG. 5 shows (A) the results of cell-mediated immunity analysis of SK-RBD-P2 and (B) the results of activity analysis of T cells and B cells.

FIG. 6 is the result showing the degree of the increase in IFN-γ secreting T cells that specifically respond to RBD. It was confirmed that the immunized substance was memorized in T cells and the T cells secreted the cytokine IFN and were activated by a stimulating antigen.

FIG. 7 shows (A) the evaluation of binding force between ACE2 and RBD-Ex1-P2 antigen and (B) the evaluation of binding force between CR3022 and an antigen for vaccine through BLI.

FIG. 8 shows the results of anti-RBD ELISA in a RBD purified stock solution.

FIG. 9 shows the results of confirming the increase in IFN-gamma secreting T cells after immunization with the antigen obtained in one embodiment of the present invention.

### MODE FOR INVENTION

Hereinafter, embodiments will be described in detail to aid understanding of the present invention. However, the embodiments according to the present invention may be modified in various forms, and the scope of the present invention should not be construed as being limited to the embodiments described below. The embodiments of the present invention are provided by way of example to aid in a specific understanding of the present invention. Embodiments of the present invention are provided to more completely explain the present invention to those with average knowledge in the field to which the present invention belongs.

1. Preparation of Construct for Antigen Expressing Using Spike Protein of SARS-Coronavirus-2

In order to prepare an antigen protein used for a vaccine production, the S gene, N gene, M gene sequences were prepared by referring to the sequence of Genbank #MN908947 Severe acute respiratory syndrome coronavirus 2 isolate Wuhan-Hu-1.

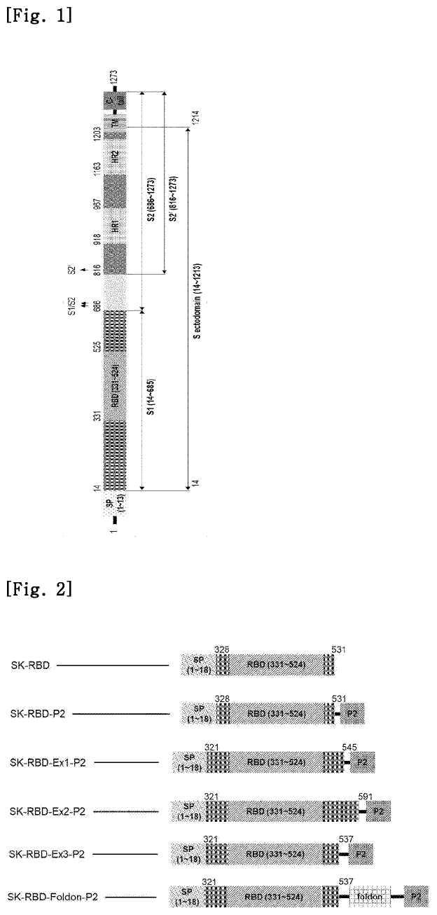

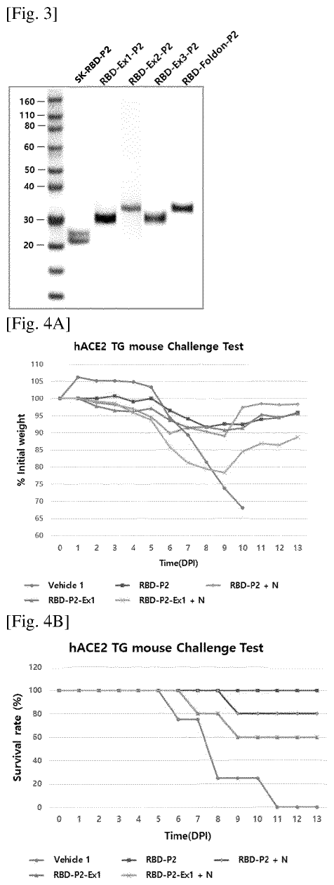

FIG. 1 shows a schematic diagram showing a structure of SARS-CoV2 spike full-length protein domain, wherein the RBD is a domain consisting of the 331st to 524th polypeptides of the full-length peptide sequence.

Researchers designed a recombinant protein antigen using the newly designed extended RBD recombinant protein (SK-RBD (SEQ ID NO: 1), or (SK-RBD-ex1, SK-RBD-ex2, and SK-RBD-ex3 expressed by SEQ ID NOs: 6, 7, and 8, respectively)), and it was illustrated in FIG. 2 in detail. SP stands for a signal protein, P2 stands for a Tetanus P2 domain (CD4 T cell epitope), and foldon stands for a foldon protein domain. Herein, the P2 domain and the foldon protein domain were each linked through a GSGSG peptide linker. The recombinant protein antigen designed in this way is represented by SEQ ID NOs: 9 to 12. A recombinant protein antigen containing a foldon domain was prepared, and represented by SEQ ID NO: 13.

Expression constructs for expressing these recombinant protein antigens were designed by adding a polynucleotide encoding an appropriate signal peptide to each expression system so that the recombinant protein can be secreted into the periplasmic region or culture medium during expression or by replacing a polynucleotide so that a heterologous signal peptide could be expressed instead of the original signal peptide. In the Spike protein, the N-terminal 1 to 13 polypeptide (MFVFLVLLPLVSS) is its own signal peptide, and in the baculovirus system, CHO cell expression system, and mammalian cell expression system expressing the recombinant protein antigen, the polypeptide replaced with the human albumin signal peptide (SEQ ID NO: 2) was allowed to be expressed, or the original signal peptide was allowed to be expressed as it is.

Table 1 below shows the characteristics of the antigen proteins obtained from the gene constructs illustrated in FIG. 2.

TABLE 1

| | | | | | | Charge | Extinction | Cys | Cyc |
|---|---|---|---|---|---|---|---|---|---|
| Section | | Residues | Length | MW | PI | at pH 7 | Coefficient | No. | % |
| Extended | SK- | 328-531 | 204 | 22.928 | 8.18 | 4.47 | 33850 | 8 | 3.9 |
| RBD | RBD(SEQ | | | | | | | | |
| | ID NO: 1) | | | | | | | | |
| | RBD- | 321~554 | 225 | 25.276 | 8.26 | 5.40 | 33850 | 9 | 4.0 |
| | ex1(SEQ ID | | | | | | | | |
| | NO: 6) | | | | | | | | |
| | RBD- | 321~591 | 271 | 30.311 | 7.69 | 2.34 | 33975 | 10 | 3.7 |
| | ex2(SEQ ID | | | | | | | | |
| | NO: 7) | | | | | | | | |
| | RBD- | 321~537 | 217 | 24.380 | 8.36 | 5.47 | 33850 | 8 | 3.7 |
| | ex3(SEQ ID | | | | | | | | |
| | NO: 8) | | | | | | | | |

PI in the above Table represents the isoelectric point. The length is the number of polypeptides, and the unit of molecular weight (MW) is kDa.

As can be seen in the above Table 1, it was found that the designed recombinant protein antigen has excellent adsorption to adjuvant and excellent refolding efficiency of the expressed protein.

In the case of SEQ ID NOs: 6, 7 and 8, the glycosylation pattern was observed as a stable single pattern upon BEV expression. On the other hand, the RBD-P2 protein obtained by the construct for RBD-P2 expression had a different glycosylation pattern, so it appeared in two bands, and the rest formed a single band. The protein formation of a single pattern with the same glycosylation means homogeneous antigenicity, and this represents important meaning for inducing immunogenicity. In addition, the N-/C-terminal portion of a protein is an important factor to be considered as the possibility of post-translational modification (PTM) is higher than that of polypeptides at other positions in the expression and purification process, and it may be related to the stability, activity and other immunorejection and the like of the protein.

The recombinant protein of the present invention was designed to stably maintain a three-dimensional structure in consideration of a single antigenicity of a protein, and its activity could be confirmed.

The structure of the extended RBD recombinant protein antigen was changed so that the N-terminus and C-terminus could be stabilized, and it was confirmed that the binding ability with ACE2 could be increased while the protein expression was maintained by this structural change.

BioLayer Interferometry (BLI) was used to evaluate the binding force of CR3022, ACE2 and the RBD protein.

In the case of SK-RBD (SEQ ID NO: 1), the protein yield was 17.1 mg/L, but in the case of RBD-P2, it was 58.5 mg/L, confirming the increased yield, and RBD-Ex1-P2 also showed a yield similar to that of RBD-P2.

On the other hand, while maintaining the protein expression yield of SK-RBD-Ex1-P2 antigen (SEQ ID NO: 10), the binding ability with ACE2 increased from 27.4 KD to 4.1 KD.

TABLE 2

| | Binding force with |
|---|---|
| Antigen protein | ACE2 protein(KD)-nM |
| Reference (Sino-RBD) | 4.4 |
| SK-RBD (SEQ ID NO: 1) | 13.3 |
| SK-RBD-P2 (SEQ ID NO: 9) | 27.4 |
| SK-RBD-Ex1-P2(SEQ ID NO: 10) | 4.1 |

2. Antigen Preparation Using Other Proteins

The N protein antigen of SEQ ID NO: 26 was prepared based on the N protein gene of the SARS-corona-2 virus.

3. Codon Optimization

The DNA sequence encoding the recombinant protein was synthesized in GenScript with codons optimized for insect cells and Chinese Hamster Ovary (CHO) cells, respectively.

The codon-optimized sequence for each expression system is as follows. The following sequence is a polynucleotide sequence.

TABLE 3

| | BEVS | CHO |
|---|---|---|
| Item | SEQ ID NO: | SEQ ID NO: |
| SK-RBD | 14 | 15 |
| SK-RBD-P2 | 16 | 17 |
| SK-RBD-Ex1-P2 | 18 | 19 |
| SK-RBD-Ex2-P2 | 20 | 21 |
| SK-RBD-Ex3-P2 | 22 | 23 |
| SK-RBD-Foldon-P2 | 24 | 25 |
| SK-S-trimer-P2 | 67 | 66 |

Further, a protein vaccine was designed with reference to the spike protein sequence (SEQ ID NOs:44 to 48) corresponding to the four popular Wuhan virus variants (B.1.1.7, B.1.351, B.1.1.248, B.1.429), and codons were optimized for the Insect and CHO expression system, and were represented by SEQ ID NOs: 49 to 64 and 66 to 67.

4. Recombinant Protein Vaccine Preparation

A recombinant protein vaccine was produced by the following procedure using baculovirus and CHO cells.

4-1. Production of Recombinant Protein Using Baculovirus Expression System

In order to express the recombinant protein (SK-RBD, SK-RBD-P2, SK-RBD-Ex1-P2, SK-RBD-Ex2-P2, SK-RBD-Ex3-P2, and SK-RBD-Foldon-P2) designed as shown in FIG. 2, and N protein with the baculovirus expression system, gene constructs represented by the codon-optimization SEQ ID NOs: 14, 16, 18, 20, 22 and 24, and SEQ ID NO: 28, respectively, were prepared. The construct gene prepared above was inserted into the transfer vector, pFastBac vector and cloned, and the gene sequence was analyzed.

The prepared plasmid was transformed into E. coli for bacmid production to prepare a recombinant bacmid, and the gene sequence was analyzed.

Recombinant baculovirus (P0) was prepared by inoculating the recombinant bacmid to Sf9 cells cultured as a monolayer for transfection and quantified by the plaque test method.

The recombinant baculovirus was infected to cultured Hi-5 cells to obtain P1 virus, and the antigen protein produced was confirmed in the supernatant.

The antigen protein produced by infecting the P1 virus into the Hi-5 cells was recovered.

The recovered recombinant protein was filtered using a filter, and the recombinant protein was purified using appropriate chromatography method (Ion Exchange, Size Exclusion and the like).

4-2. Production of Recombinant Protein Using CHO Cell Expression

In order to express the recombinant protein (SK-RBD, SK-RBD-P2, SK-RBD-Ex1-P2, SK-RBD-Ex2-P2, SK-RBD-Ex3-P2, and SK-RBD-Foldon-P2) designed as shown in FIG. 2, and N protein with the baculovirus expression system, gene constructs represented by the codon-optimization SEQ ID NOs: 15, 17, 19, 21, 23 and 25, and SEQ ID NO: 29, respectively, were prepared.

The synthesized gene was inserted into an expression vector and cloned, and the gene sequence was analyzed.

The recombinant plasmid was transfected into CHO cells for protein production (CHO K-1 cell line).

The transfected cells expressing the recombinant protein were identified using antibiotics.

The identified transfected CHO cells were mass-cultured and the recombinant protein was recovered.

The recovered recombinant protein was filtered using a filter, and the recombinant protein was purified using appropriate chromatography method (Ion Exchange, Size Exclusion and the like).

4-3. Recombinant Protein Identification and Quantification

The expression of the recombinant protein was confirmed using SDS-PAGE and Western blot method. The recombinant protein was quantified using a basic total protein quantification method (Lowry method, BCA method and the like).

5. Recombinant Antigen Protein Evaluation

5-1. Immunogenicity Test

The purified recombinant protein was combined with an adjuvant (e.g., Aluminum hydroxide) and injected into an animal model 2 to 3 times at intervals of 2 to 3 weeks. Safety was confirmed by measuring changes in body weight and body temperature. 2 to 3 weeks after the final injection, serum isolated from whole blood and splenocytes were obtained.

5-2. Protection Test

The purified recombinant protein was combined with an adjuvant (e.g., Aluminum hydroxide) and injected into an animal model 2 to 3 times at intervals of 2 to 3 weeks. 2 to 3 weeks after the final injection, the animal was infected with a lethal amount of wild type SARS-Coronavirus-2 virus. For one week after the infection, virus shedding was evaluated in the nasal cavity, airways, organs and the like. For two weeks after the infection, changes in body weight and body temperature, survival rate and the like were evaluated.

5-3. Immunogenicity Evaluation Analysis

For immunogenicity evaluation analysis, IgG ELISA assay was used. An antigen for coating (RBD, 51, S2, N and the like) was coated on a 96 well-plate, and the plate was blocked with a blocking buffer. The sample (serum) was reacted on the plate. An IgG detection antibody was reacted on the plate. A substrate buffer was added to develop color, and the absorbance was measured.

5-4. Pseudovirus Preparation

An S protein gene of SARS-Coronavirus-2 was cloned into an expression vector. A reporter gene was cloned into a transfer vector. The two genes were transfected into cells for pseudovirus production to prepare a pseudovirus expressing the reporter protein.

5-5. Neutralizing Antibody Titer Evaluation

The serially diluted sample (serum) was reacted with the pseudovirus. Cells for infection cultured in a 96 well-plate (Vero E6 and the like) were infected with the reacted pseudovirus and cultured. After 4 to 6 hours, it was washed with PBS and replaced with a new medium. After culturing for 24 to 72 hours, the expression level of the reporter protein was compared to evaluate the neutralizing antibody titer.

5-6. Cell-Mediated Immunity Evaluation

An anti-IFN-γ antibody was coated on a 96 well-plate. The plate was blocked with a blocking buffer. Splenocytes and a stimulating antigen (Stimulate) were added thereto and cultured for 24 to 36 hours. An Interferon-gamma detection antibody was reacted, and a substrate was added and reacted. Immune cells were evaluated using an ELISPOT reader.

For the analysis of immune characteristics, an immune cell-specific antibody and a cytokine antibody were reacted with the isolated splenocytes for 2 hours. T cell distribution and cytokine expression rate were measured through flow cytometry.

5-7. Antigenicity Evaluation of Antigen for Vaccine

BioLayer Interferometry (BLI) was used to evaluate the binding force with CR3022. CR3022 is a human monoclonal antibody against the recombinant SARS-CoV-2 Spike Glycoprotein 51. (Abcam, CAT #: ab273073)

BLI measures the affinity constant KD value (Kdis/Kon) through association and dissociation between an antibody and an antigen, and the smaller value, the higher affinity. Coronal 9 S-specific antibody was immobilized on ProA sensor chip (ForteBio) using Octet K2. The association was measured by dipping the sensor chip into an antigen sample diluted 2-fold from 100 nM, and the dissociation was measured by dipping into a well containing only a kinetic buffer. The data obtained by subtracting the reference from the result value was analyzed by fitting thereto to a 1:1 binding model using Octet Data Analysis software (11.0).

Enzymatic immunoassay was performed to demonstrate the biological activity and structural robustness of the antigen. Immune specific response was confirmed using the RBD protein as a main antigen of the recombinant Coronal 9 vaccine manufactured by our company and anti-SARS-CoV-2 neutralizing antibody, Human IgG1 (Acrobiosystems, Cat No. SAD-S53) neutralizing antibody or SARS-CoV-2 Spike neutralizing antibody, Mouse Mab (SinoBio, Cat No. MM57).

6. Immunogenicity Test Result Through Total Antibody Titer/Neutralizing Antibody Titer Analysis 6-1. Result of Comparison Test of Immunogenicity Between SK-RBD and SK-RBD-P2 Using BALB/c

A 6-week-old female mouse was immunized with immunogenic substances, SK-RBD (SEQ ID NO: 1) and SK-RBD-P2 (SEQ ID NO: 9) by intramuscular injection (IM) 2 times at 3 weeks intervals. Then, blood was collected, serum was isolated and immunogenicity was analyzed. As a result of the analysis, it was confirmed that the antibody titer was formed by SK-RBD (SEQ ID NO: 1) and SK-RBD-P2 (SEQ ID NO: 9). Groups 1 and 2 were administered with PBS and aluminum hydroxide (=Alum. H), respectively, in the same amount as in groups 3 to 6 without administration of the antigen. As can be seen in Table 4, both groups showed high IgG antibody titer at weeks 6 and 8, but SK-RBD-P2 (SEQ ID NO: 9) showed saturation pattern at week 8. The total IgG value of the immune sample of week 8 was 2581 in SK-RBD (SEQ ID NO: 1) and 136462 in SK-RBD-P2 (SEQ ID NO: 9). The total antibody value induced by SK-RBD-P2 (SEQ ID NO: 9) was more than 5 times higher antibody titer, demonstrating better immunogenicity. N protein specific IgG antibody was also found in groups 4 and 6 immunized together with the N protein (SEQ ID NO: 26) (Table 4).

TABLE 4
Analysis of total antibody titer and neutralizing antibody titer of mouse
immunized with RBD and RBD-P2 (BALB/c mouse)

| | | Antigen | | | | | | | | |
|---|---|---|---|---|---|---|---|---|---|---|
| | | amount | Subject | | RBD-specific total IgG | | | N-specific total IgG | | |
| No. | Antigen | (μg/dose) | No. | Adjuvant | 4 w | 6 W | 8 W | 4 W | 6 W | 8 W |
| 1 | Vehicle 1 | 0 | 10 | PBS | 67 | 25 | 25 | 189 | 128 | 385 |
| 2 | Vehicle 2 | 0 | 10 | Alum. H | 25 | 25 | 25 | 70 | 83 | 276 |
| 3 | SK-RBD- | 10 | 10 | Alum. H | 26796 | 146153 | 136462 | 73 | 81 | 229 |
| | P2(SEQ ID | | | | | | | | | |
| | NO: 9) | | | | | | | | | |
| 4 | SK-RBD- | 10 + 1 | 10 | Alum. H | 2923 | 8291 | 89642 | 560185 | 3318538 | 486428 |
| | P2(SEQ ID | | | | | | | | | |
| | NO: 9) + | | | | | | | | | |
| | N(SEQ ID | | | | | | | | | |
| | NO: 26) | | | | | | | | | |
| 5 | SK- | 10 | 10 | Alum. H | 3916 | 7041 | 25182 | 108 | 110 | 210 |
| | RBD(SEQ | | | | | | | | | |
| | ID NO: 1) | | | | | | | | | |
| 6 | SK- | 10 + 1 | 10 | Alum. H | 3489 | 9029 | 12076 | 154435 | 317386 | 301893 |
| | RBD(SEQ | | | | | | | | | |
| | ID NO: 1) + | | | | | | | | | |
| | N(SEQ ID | | | | | | | | | |
| | NO: 26) | | | | | | | | | |

6-2. Analysis of Total Antibody Titer and Neutralizing Antibody Titer of Mouse Immunized with SK-RBD-P2 (SEQ ID NO: 9) (BALB/c Mouse)

A 6-week-old female mouse was immunized with SK-RBD-P2 (SEQ ID NO: 9) and N (SEQ ID NO: 26) antigens by IM 2 times at 3 weeks intervals. Then, blood was collected, serum was isolated and immunogenicity was analyzed. The total antibody titer was measured by performing ELISA with the mouse immune serum at week 5 and week 6. As a result of analysis, as the amount of the administered antigen SK-RBD-P2 (SEQ ID NO: 9) increased (5, 10, 30 μg), the antibody titer increased dose-dependently. It was confirmed that N-specific antibody titer was formed in the serum immunized together with the N antigen. Looking at the groups 3 and 6 in Table 5 below, when the N protein antigen is administered together, there is no difference in the value of neutralizing antibody, but the ability to induce cell-mediated immunity is excellent. Therefore, this enables effective protection in the early stage of virus infection.

TABLE 5
Analysis of total antibody titer and neutralizing antibody titer of mouse
immunized with SK-RBD-P2 (SEQ ID NO: 9) (BALB/c mouse)

| | | | | | | | | | Neutralizing |
|---|---|---|---|---|---|---|---|---|---|
| | | Antigen | | | Total IgG titer | | | | antibody |
| | | amount | Subject | | 4 w | 6 w | 4 w | 6 w | titer |
| No. | Antigen | (μg/dose) | No. | Adjuvant | RBD-specific | | N-specific | | PBNA₅₀ |
| 1 | Vehicle 1 | 0 | 5 | PBS | 25 | 25 | 114 | 223 | 10 |
| 2 | SK-RBD-P2(SEQ | 5 | 5 | Alum. H | 1666 | 2536 | 159 | 467 | ND |
| | ID NO: 9)-5 | | | | | | | | |
| 3 | SK-RBD-P2(SEQ | 10 | 5 | Alum. H | 7323 | 20336 | 252 | 781 | 10 |
| | ID NO: 9)-10 | | | | | | | | |
| 4 | SK-RBD-P2(SEQ | 30 | 5 | Alum. H | 30499 | 215966 | 252 | 781 | 320 |
| | ID NO: 9)-30 | | | | | | | | |
| 5 | SK-RBD-P2(SEQ | 5 + 0.5 | 5 | Alum. H | 100 | 313 | 84210 | 566945 | 20 |
| | ID NO: 9)-5 + | | | | | | | | |
| | N(SEQ ID NO: 26)-0.5 | | | | | | | | |
| 6 | SK-RBD-P2(SEQ | 10 + 1 | 5 | Alum. H | 1504 | 7044 | 86971 | 877576 | 10 |
| | ID NO: 9)-10 + | | | | | | | | |
| | N(SEQ ID NO: 26)-1 | | | | | | | | |
| 7 | SK-RBD-P2(SEQ | 30 + 3 | 5 | Alum. H | 7399 | 27697 | 176099 | 1394533 | 160 |
| | ID NO: 9)-30 + | | | | | | | | |
| | N(SEQ ID NO: 26)-3 | | | | | | | | |

*ND : Not Detected

6-3. Analysis of Total Antibody Titer and Neutralizing Antibody Titer of Mouse Immunized with RBD-Ex1-P2 (SEQ ID NO: 10) and RBD-Ex2-P2 (SEQ ID NO: 11) (BALB/c Mouse)

6-Week-old female BALB/c mouse was prepared, and immunized to the muscle with 0.1 ml of RBD-Ex1-P2 (SEQ ID NO: 10), RBD-Ex2-P2 (SEQ ID NO: 11) and N (SEQ ID NO: 26) proteins mixed with aluminum hydroxide 2 times at 3 weeks intervals. Then, blood was collected, serum was isolated and analyzed. As a result of the analysis, it was confirmed that RBD-Ex1-P2 (SEQ ID NO: 10) and RBD-Ex2-P2 (SEQ ID NO: 11) formed RBD-specific antibody titer and N-specific antibody titer, and as the amount of the administered antigen increased (5, 10, 30 μg), the antibody titer increased dose-dependently. Further, when N was administered together in an amount of 1/10, the RBD-specific IgG antibody titer tended to be slightly lower, but the neutralizing antibody titer was induced to the same level. RBD-specific IgG antibody titer and neutralizing antibody titer were shown in the group immunized with Alum+CpG adjuvant rather than Alum alone. As the CpG, Dynavax's brand name CpG 1018 adjuvant was used.

When 10 μg was administered, in the case of the alum adjuvant, the RBD-specific antibody titer was 4221, and the neutralizing antibody titer was similar to that of the vehicle, so it was hardly induced, but in the case of the alum+CpG, the RBD specific antibody titer was 5389108 and the neutralizing antibody titer was 320 or higher, which were very high (Table 6).

TABLE 6
Analysis of total antibody titer and neutralizing antibody titer of mouse
immunized with RBD-Ex1-P2 (SEQ ID NO: 10) and RBD-Ex2-P2 (SEQ ID NO: 11)
(BALB/c mouse)

| | | Antigen | | | Total IgG titer | | | | |
|---|---|---|---|---|---|---|---|---|---|
| | | amount | Subject | | 3 w | 5 w | 3 w | 5 w | |
| No. | Antigen | (ug/dose) | No. | Adjuvant | RBD-specific | | N-specific | | PBNA₅₀ |
| 1 | Vehicle 1 | 0 | 5 | Alum | 25 | 25 | 23995 | 592 | 40 |
| 2 | RBD-Ex1-P2(SEQ | 5 | 5 | Alum. H | 25 | 2706 | 123 | 119 | 10 |
| | ID NO: 10)-5 | | | | | | | | |
| 3 | RBD-Ex1-P2(SEQ | 10 | 5 | Alum. H | 25 | 4221 | 151 | 127 | 40 |
| | ID NO: 10)-10 | | | | | | | | |
| 4 | RBD-Ex1-P2(SEQ | 30 | 5 | Alum. H | 1207 | 104476 | 177 | 353 | 80 |
| | ID NO: 10)-30 | | | | | | | | |
| 5 | RBD-Ex1-P2(SEQ | 5 + 0.5 | 5 | Alum. H | 74 | 3686 | 930 | 542933 | 0 |
| | ID NO: 10)-5 + | | | | | | | | |
| | N(SEQ ID NO: 26)-0.5 | | | | | | | | |
| 6 | RBD-Ex1-P2(SEQ | 10 + 1 | 5 | Alum. H | 25 | 2174 | 3460 | 282976 | 10 |
| | ID NO: 10)-10 + | | | | | | | | |
| | N(SEQ ID NO: 26)-1 | | | | | | | | |
| 7 | RBD-Ex1-P2(SEQ | 30 + 3 | 5 | Alum. H | 61 | 29738 | 15264 | 388091 | 160 |
| | ID NO: 10)-30 + | | | | | | | | |
| | N(SEQ ID NO: 26)-3 | | | | | | | | |
| 8 | RBD-Ex2-P2(SEQ | 5 | 5 | Alum. H | 25 | 1104 | 98 | 25 | 0 |
| | ID NO: 11)-5 | | | | | | | | |
| 9 | RBD-Ex2-P2(SEQ | 10 | 5 | Alum. H | 25 | 2839 | 137 | 60 | 0 |
| | ID NO: 11)-10 | | | | | | | | |
| 10 | RBD-Ex2-P2(SEQ | 30 | 5 | Alum. H | 2600 | 43961 | 161 | 25 | 80 |
| | ID NO: 11)-30 | | | | | | | | |
| 11 | RBD-Ex2-P2(SEQ | 5 + 0.5 | 5 | Alum. H | 167 | 25 | 772 | 61379 | 0 |
| | ID NO: 11)-5 + | | | | | | | | |
| | N(SEQ ID NO: 26)-0.5 | | | | | | | | |
| 12 | RBD-Ex2-P2(SEQ | 10 + 1 | 5 | Alum. H | 58 | 704 | 2640 | 536857 | 0 |
| | ID NO: 11)-10 + | | | | | | | | |
| | N(SEQ ID NO: 26)-1 | | | | | | | | |
| 13 | RBD-Ex2-P2(SEQ | 30 + 3 | 5 | Alum. H | 25 | 6329 | 5593 | 1314727 | 80 |
| | ID NO: 11)-30 + | | | | | | | | |
| | N(SEQ ID NO: 26)-3 | | | | | | | | |
| 14 | RBD-Ex1-P2(SEQ | 10 | 5 | Alum. | 112478 | 5389108 | 121 | 115 | >320 |
| | ID NO: 10)-10 | | | H + CpG | | | | | |
| 15 | RBD-Ex1-P2(SEQ | 10 + 1 | 5 | Alum. | 13988 | 900862 | 198650 | 235167 | >320 |
| | ID NO: 10)-10 + | | | H + CpG | | | | | |
| | N(SEQ ID NO: 26)-1 | | | | | | | | |

Through the above result, it was found that the recombinant protein antigen of the group 14 and the like was excellent in generating a neutralizing antibody. In addition, when the N protein was administered together, it was found that it was effective in inducing cell-mediated immunity response required for initial virus protection as well as the generation of a neutralizing antibody.

6-4. Total Antibody Titer and Neutralizing Antibody Titer Analysis According to Ratio of RBD-Ex1-P2 (SEQ ID NO: 10) and N (SEQ ID NO: 26) (BALB/c Mouse)

A 6-week-old female mouse was immunized with an antigen by IM 2 times at 3 weeks intervals. Then, blood was collected, serum was isolated and immunogenicity was analyzed. As a result of analysis, it was confirmed that the antibody titer was formed by the RBD-Ex1-P2 (SEQ ID NO: 10) and the N (SEQ ID NO: 26). In order to confirm the difference in immunogenicity according to the N protein injection, the N (SEQ ID NO: 26) antigen was immunized with two doses of 1/10 and 1/50 of the amount of the RBD-Ex1-P2 (SEQ ID NO: 10) antigen, and the RBD-specific antibody titer, the N-specific antibody titer and the neutralizing antibody titer were analyzed. As a result of analysis, when the N (SEQ ID NO: 26) was administered at a level of 1/10 of the amount of the RBD-Ex1-P2 (SEQ ID NO: 10) antigen, the RBD-specific antibody titer tended to decrease slightly, but the neutralizing antibody was similar or slightly increased, and when administered at a level of 1/50, both the RBD-specific antibody and the neutralizing antibody titer were significantly increased. When the RBD-Ex1-P2 (SEQ ID NO: 10) was administered alone, the RBD-specific antibody titer and the neutralizing antibody titer increased in a dose-dependent manner in the range of 5 to 50 ug, but when the N (SEQ ID NO: 26) was co-administered at a level of 1/50 of the amount of the RBD-Ex1-P2 (SEQ ID NO: 10) antigen, in the case of administering 30 ug of the RBD-Ex1-P2 (SEQ ID NO: 10), higher level of the RBD-specific antibody and neutralizing antibody titer were induced than the case of administering 50 ug of the RBD-Ex1-P2 (SEQ ID NO: 10).

TABLE 7
Analysis of total antibody titer and neutralizing antibody titer of mouse
immunized with combination of RBD-Ex1-P2 (SEQ ID NO: 10) and N (SEQ ID NO:
26) (BALB/c mouse)

| | | Antigen | | | Total IgG titer | | | | |
|---|---|---|---|---|---|---|---|---|---|
| | | amount | Subject | | 3 w | 6 w | 3 w | 6 w | |
| No. | Antigen | (ug/dose) | No. | Adjuvant | RBD-specific | | N-specific | | PBNA₅₀ |
| 1 | Vehicle 1 | 0 | 5 | Alum. H | 30 | 25 | 33 | 25 | 0 |
| 2 | RBD-Ex1-P2(SEQ | 30 | 5 | Alum. H | 121 | 57364 | 39 | 25 | 80 |
| | ID NO: 10)-30 | | | | | | | | |
| 3 | RBD-Ex1-P2(SEQ | 30 + 3 | 5 | Alum. H | 35 | 52590 | 4089 | 190572 | 320 |
| | ID NO: 10)-30 + | | | | | | | | |
| | N(SEQ ID NO: 26)- | | | | | | | | |
| | 3 | | | | | | | | |
| 4 | RBD-Ex1-P2(SEQ | 30 + 0.6 | 5 | Alum. H | 498 | 109455 | 1503 | 84553 | 1280 |
| | ID NO: 10)-30 + | | | | | | | | |
| | N(SEQ ID NO: 26)- | | | | | | | | |
| | 0.6 | | | | | | | | |

6-5. Analysis of Total Antibody Titer of RBD-Ex1-P2 (SEQ ID NO: 10) and N (SEQ ID NO: 26) (SD-Rat)

A 7-week-old female rat was immunized with an antigen by IM 2 times at 3 weeks intervals. Then, blood was collected, serum was isolated and immunogenicity was analyzed. As a result of analysis, it was confirmed that the RBD-Ex1-P2 (SEQ ID NO: 10)-specific antibody titer and the N (SEQ ID NO: 26)-specific antibody titer were formed. In order to confirm the difference in immunogenicity according to the co-administered N protein injection, the total antibody titer and the neutralizing antibody were analyzed with the serum of the fully immunized mouse. As a result of analysis, it was confirmed that the RBD-specific antibody titer and N-specific IgG antibody titer were formed as shown in the graph below, and it was confirmed that the highest level of the total antibody titer was formed in the group 5 immunized with the RBD-Ex1-P2 (SEQ ID NO: 10) and the N (SEQ ID NO: 26) proteins at 50 ug and 5 ug, respectively.

TABLE 8
Analysis of total antibody titer of rat immunized with combination
of RBD-Ex1-P2 (SEQ ID NO: 10) and N (SEQ ID NO: 26)

| | | Antigen amount | Subject | | RBD-specific | N-specific |
|---|---|---|---|---|---|---|
| No. | Antigen | (ug/dose) | No. | Adjuvant | IgG titer | IgG titer |
| 1 | Vehicle 1 | 0 | 10 | Alum. H | 28 | 172 |
| 2 | RBD-Ex1-P2(SEQ ID | 30 | 10 | Alum. H | 6875 | 71 |
| | NO: 10)-30 | | | | | |
| 3 | RBD-Ex1-P2(SEQ ID | 50 | 10 | Alum. H | 13145 | 71 |
| | NO: 10)-50 | | | | | |
| 4 | RBD-Ex1-P2(SEQ ID | 30 + 3 | 10 | Alum. H | 6392 | 16220 |
| | NO: 10)-30 + N | | | | | |
| | (SEQ ID NO: 26)-3 | | | | | |
| 5 | RBD-Ex1-P2(SEQ ID | 50 + 5 | 10 | Alum. H | 31943 | 107793 |
| | NO: 10)-50 + N | | | | | |
| | (SEQ ID NO: 26)-5 | | | | | |

6-6. Analysis of Cellular Immunogenicity of RBD-Ex1-P2 (SEQ ID NO: 10) and N (SEQ ID NO: 26) (SD-Rat)

In order to confirm the induction of cellular immunogenicity of a rat in the same group as in Table 8, the spleen was isolated from the fully immunized rat and ELISPot was performed. As a result of analysis, an increase in the number of IFN-gamma secreting T cell specifically responding to the RBD-Ex1-P2 (SEQ ID NO: 10) antigen stimulation was confirmed in the immune groups (G2 to G5). Further, an increase in the number of IFN-gamma secreting T cell specifically responding to the stimulating antigen N (SEQ ID NO: 26) was confirmed in the groups G4 and G5 immunized with the N (SEQ ID NO: 26) antigen.

6-7. Analysis of Total Antibody Titer and Neutralizing Antibody Titer of Transgenic Mouse Immunized with RBD-Ex1-P2 (SEQ ID NO: 10) (hACE2 TG Mouse)

The total antibody titer was measured by performing ELISA with the immune serum of week 5 and week 6 from a TG mouse expressing a Human ACE2 gene. As a result of analysis, it was confirmed that the RBD-specific antibody titer was formed at week 6 at the level of 136077 as shown in the graph below. PBNA neutralizing antibody titer analysis was performed with the serum of week 6 from the mouse immunized with the RBD-Ex1-P2 (SEQ ID NO: 10) antigen. It was confirmed that in the hACE2 TG mouse susceptible to the wild-type SARS-CoV-2, the serum at week 6 showed PBNA₅₀ value of 320 and the neutralizing antibody titer was formed.

TABLE 9
Analysis of total antibody titer and neutralizing antibody
titer of transgenic mouse immunized with
SK-RBD-P2 (SEQ ID NO: 9) (25 hACE2 TG mice)

| | | Antigen | | | RBD specific | | |
|---|---|---|---|---|---|---|---|
| | | amount | Subject | | total IgG | | |
| No. | Antigen | (ug/dose) | No. | Adjuvant | 5 w | 6 w | PBNA₅₀ |
| 1 | Vehicle 1 | 0 | 5 | Alum. H | 25 | 25 | 20 |
| 2 | SK-RBD- | 20 | 5 | Alum. H | 28307 | 161692 | 640 |
| | P2(SEQ ID | | | | | | |
| | NO: 9) | | | | | | |

TABLE 10
Analysis of total antibody titer and neutralizing antibody titer of transgenic
mouse immunized with RBD-Ex1-P2 (SEQ ID NO: 10) antigen (hACE2 TG mouse)

| | | Antigen | | | RBD specific | | |
|---|---|---|---|---|---|---|---|
| | | amount | Subject | | total IgG | | PBNA₅₀ |
| Group | Antigen | (ug/dose) | No. | Adjuvant | 5 w | 6 w | (Pseudovirus) |
| 1 | Vehicle | 0 | 5 | Alum. H | 25 | 25 | ND* |
| 2 | RBD-Ex1-P2 | 20 | 5 | Alum. H | 22158 | 136077 | 320 |
| | (SEQ ID NO: 10) | | | | | | |

*ND: Not Detected

TABLE 11
Analysis of challenge test result of transgenic mouse immunized with
RBD-Ex1-P2 (SEQ ID NO: 10) (25 hACE2 TG mice)

| | | Antigen | | |
|---|---|---|---|---|
| | | amount | Subject | |
| No. | Antigen | (ug/dose) | No. | Adjuvant |
| 1 | Vehicle 1 | 0 | 5 | Alum. H |
| 2 | SK-RBD-P2 (SEQ ID NO: 9) | 20 | 5 | Alum. H |
| 3 | RBD-Ex1-P2(SEQ ID NO: | 20 | 5 | Alum. H |
| | 10) | | | |

After nasal infection with the wild type SARS-CoV-2 virus (NCCP 43326) in an amount of 5×10⁴ pfu/mouse, weight change and death rate were investigated for 12 days. As a result, in the case of vehicle 1 group, one died on day 6, two died on day 8, and one died on day 11, resulting in a total of 100% deaths excluding one without infection. However, 80% of the animals of the group administered with the RBD-P2 and all animals of the group administered with the RBD-Ex1-P2 vaccine survived. In other words, the survival rate was 80% or more. Therefore, it was confirmed that the recombinant protein antigen of the present invention could act as an excellent immunogen. Further, in the change of body weight after infection, in the vaccine group, it was showed an aspect that the body weight decreased within 20% and then gradually recovered, but in the vehicle group, death occurred with a rapid weight loss of about 30%. The vaccine was 100% protective in the TG mouse susceptibly modified to SARS-CoV-2 virus (FIG. 4a and FIG. 4b).

7. Analysis of Mouse Cell-Mediated Immunity Result

7-1. Result of Cellular Immunogenicity Analysis of BALB/c Mouse Immunized with RBD-P2

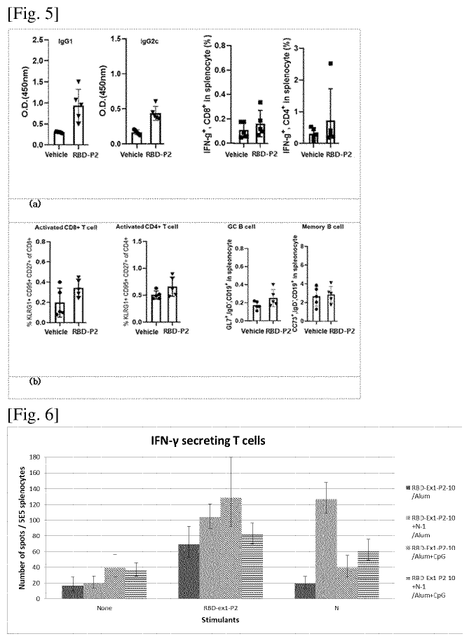

In an animal experiment using C57BL/6, IgG subtype analysis and cell-mediated immunity induction pattern analysis were performed. As a result of performing isotype antibody analysis of IgG1 and IgG2c in the serum, it was confirmed that both IgG1 and IgG2 subtype antibody titers were increased in the serum injected with the RBD-P2 antigen, and the tendency of increasing CD4+, CD8+ T cells was confirmed through FACS analysis (FIG. 5A).

Activated CD8+ cells and CD4+ cells were analyzed for analysis of T cell immunity and B cell immunity. As shown in FIG. 11, RBD-specific T cell activity tended to increase in the RBD-P2 immune group compared to the vehicle group. Further, the pattern of B cell increase in the germinal center was confirmed (FIG. 5B).

7-2. Result of Cellular Immunogenicity Analysis of BALB/c Mouse Immunized with RBD-Ex1-P2

In order to confirm the induction of cellular immunogenicity in the immunization experiment using BALB/c, splenocytes of some subjects were isolated at week 3 after the second immunization and ELISPot measuring IFN-γ secreting T cell was performed. As a result, it was confirmed that the number of T cells responding specifically to the RBD-Ex1-P2 protein antigen was significantly increased in the vaccine administered group (Table 12, FIG. 6).

TABLE 12
Mouse immunogenicity group information (RBD-Ex1-P2
immunized mouse (BALB/c mouse) test)

| | | Antigen | | |
|---|---|---|---|---|
| | | amount | Subject | |
| Group | Antigen | (ug/dose) | No. | Adjuvant |
| 1 | Vehicle | 0 | 5 | Alum. H |
| 2 | RBD-Ex1-P2(SEQ ID NO: 10)- | 10 | 5 | Alum. H |
| | 10 | | | |
| 3 | RBD-Ex1-P2(SEQ ID NO: 10)- | 10 | 5 | Alum. H |
| | 10 + N (SEQ ID NO: 26)-1 | | | |
| 4 | RBD-Ex1-P2 | 10 | 5 | Alum. H + |
| | (SEQ ID NO: 10)-10 | | | CpG |
| 5 | RBD-Ex1-P2(SEQ ID NO: 10)- | 10 | 5 | Alum. H + |
| | 10 + N (SEQ ID NO: 26)-1 | | | CpG |

8. Binding Force Evaluation Result

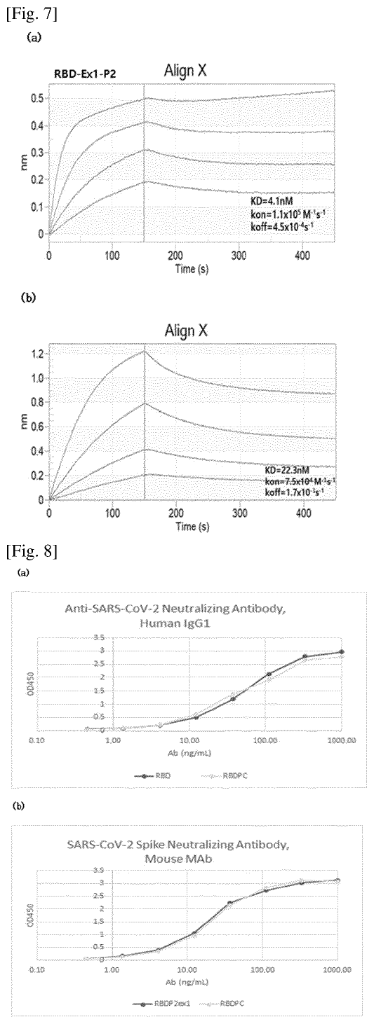

The Bio-layer Interferometry (BLI) principle was used to check whether the prepared antigen binds well to its receptor, ACE2. The binding force between the antigen for a vaccine and ACE2 (FIG. 7A) and CR3022 (FIG. 7B) was evaluated. It was confirmed that the binding force was the following Dissociation constant (KD) values, which was similar to the binding force (KD=4.4 nM) of the reference RBD (sino, Cat. 40592-V08B, Sino-RBD), and it was confirmed that the RBD-Ex1-P2 (SEQ ID NO: 10) had no problem with the ACE2 binding site and no problem with the binding function (FIG. 7).

Specifically, FIG. 7 shows (A) the evaluation of binding force between ACE2 and RBD-Ex1-P2 antigen and (B) the evaluation of binding force between CR3022 and an antigen for vaccine through BLI. The terminal structure of the extended RBD of the present invention was stabilized when compared to the RBD before the modification. Due to this, the protein expression level increased and the binding with the ACE2 increased, resulting in increased cell-mediated immunity. Low KD value indicates excellent binding (KD=Koff/Kon), and as shown in the result of FIG. 7A, it was confirmed that the KD value was higher than that of CR3022, so that the binding force with the ACE2 was excellent.

The RBD protein, which is the main antigenic site of the RBD-Ex1-P2 (SEQ ID NO: 10), was immunospecifically confirmed through enzyme immunoassay. By confirming the protein binding using a neutralizing antibody, it was confirmed that there was no abnormality in the biological activity and immunological activity of the RBD-Ex1-P2 (SEQ ID NO: 10) antigen (FIG. 8). RBDPC stands for Sino biological RBD reference (SinoBiologinal, 40592-VO8H).

Through this, synthetic sequence and information, protein expression confirmation, protein isolation and purification, and recombinant protein vaccine candidates were secured.

This can induce a sufficient antibody and protective immunity to prevent corona infection.

9. Result of Comparison Test of Immunogenicity of SK-RBD-P2 (SEQ ID NO: 9), SK-RBD-P2 (SEQ ID NO: 9)+N(SEQ ID NO: 26), S-Trimer-P2 (SEQ ID NO: 65)+N(SEQ ID NO: 26) Using BALB/c

A 6-week-old female mouse was immunized with SK-RBD-P2 (SEQ ID NO: 9), SK-RBD-P2 (SEQ ID NO: 9)+N(SEQ ID NO: 26), S-Trimer-P2 (SEQ ID NO: 65)+N(SEQ ID NO: 26) antigen by IM 2 times at 2 weeks intervals. Then, blood was collected, serum was isolated and immunogenicity was analyzed. As a result of analysis, it was confirmed that RBD protein-specific antibody titer was formed in all immune group (G2-G4). The N protein-specific antibody showed high IgG titer at week 4 in both immune groups (G3, G4) and demonstrated excellent immunogenicity (Table 13).

TABLE 13
Analysis of total antibody titer and neutralizing antibody titer of mouse
immunized with SK-RBD-P2 (SEQ ID NO: 9), SK-RBD-P2 (SEQ ID NO: 9) + N(SEQ
ID NO: 26), S-Trimer-P2 (SEQ ID NO: 65) + N(SEQ ID NO: 26) (BALB/c mouse)

| | | Antigen | | | Total IgG titer | | | | |
|---|---|---|---|---|---|---|---|---|---|
| | | amount | Subject | | 2 w | 4 w | 2 w | 4 w | |
| No. | Antigen | (ug/dose) | No. | Adjuvant | RBD-specific | | N-specific | | PBNA₅₀ |
| 1 | Vehicle 1 | 0 | 5 | Alum. H | 25 | 25 | 233 | 190 | ND |
| 2 | SK-RBD- | 10 | 5 | Alum. H | 52 | 12564 | 109 | 67 | ND |
| | P2(SEQ ID | | | | | | | | |
| | NO: 9) | | | | | | | | |
| 3 | SK-RBD-P2 | 10 + 1 | 5 | Alum. H | 25 | 5423 | 1321 | 152911 | ND |
| | (SEQ ID | | | | | | | | |
| | NO: 9) + | | | | | | | | |
| | N(SEQ ID | | | | | | | | |
| | NO: 26) | | | | | | | | |
| 4 | S-Trimer-P2 | 10 + 1 | 5 | Alum. H | 25 | 730 | 291 | 3949 | 40 |
| | (SEQ ID | | | | | | | | |
| | NO: 65) + | | | | | | | | |
| | N(SEQ ID | | | | | | | | |
| | NO: 26) | | | | | | | | |

10. Analysis of Cellular Immunogenicity of SK-RBD-P2 (SEQ ID NO: 9), SK-RBD-P2 (SEQ ID NO: 9)+N(SEQ ID NO: 26), S-Trimer-P2 (SEQ ID NO: 65)+N(SEQ ID NO: 26) (Balb/c Mouse)

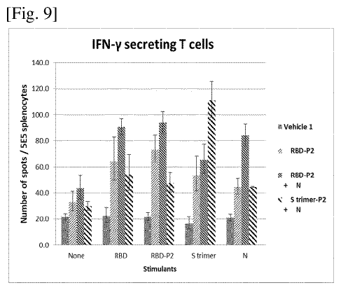

In order to confirm the induction of cellular immunogenicity in the mouse immunized with the antigen of Table 13, the spleen was isolated from the fully immunized mouse and ELISPot was performed. As a result of analysis, the increase in IFN-gamma-secreting T cells specifically responding to the immunized antigen, N-peptide, and p2 peptide stimulation in the immune group (No. 2 to 4 above) excluding the vehicle was confirmed. The results are shown in FIG. 9. As can be seen from these results, the antigens of the present invention exhibited excellent effects of cell-mediated immunity response.

11. Analysis of Total Antibody Titer and Neutralizing Antibody Titer of Transgenic Rat Immunized with SK-RBD (SEQ ID NO: 1), S-Trimer-P2 (SEQ ID NO: 65), N(SEQ ID NO: 26), Respectively

As a result of analysis of the serum of the RBD immunized groups of Table 14, the RBD-specific IgG antibody titer increased on Days 14, 28 and 43, and decreased on Day 57, compared to the vehicle group (G1). In the case of the S-Trimer-P2 (SEQ ID NO: 65), the S-Trimer-P2 (SEQ ID NO: 65)-specific antibody titer increased until Day 43, and then the antibody titer decreased, compared to the vehicle group (G1). As a result of analyzing the generation of the N-specific antibody in the serum of the groups immunized together with the N (G3, G5), the antibody titer increased by 227-2106 times until Day 43, compared to the vehicle group (G1), and then saturated.

TABLE 14

| | | | IgG Titer | | | | |
|---|---|---|---|---|---|---|---|
| | Group | | Day | Day | Day | Day | Day |
| Antigen | No. | Group No. | 0 | 14 | 28 | 43 | 57 |
| SK RBD(SEQ | G1 | Vehicle | 25 | 25 | 29 | 25 | 25 |
| ID NO: 1) | G2 | SK-RBD (SEQ ID NO: 1) | 25 | 52 | 6373 | 166915 | 77863 |
| | G3 | SK-RBD (SEQ ID NO: 1) + | 25 | 83 | 4663 | 169083 | 74617 |
| | | N(SEQ ID NO: 26) | | | | | |
| S-Trimer- | G1 | Vehicle | 25 | 25 | 33 | 38 | 37 |
| P2(SEQ ID | G4 | S-Trimer-P2 (SEQ ID NO: 65) | 25 | 1094 | 39042 | 116311 | 99781 |
| NO: 65) | G5 | S-Trimer-P2 (SEQ ID NO: 65) + | 25 | 1887 | 53134 | 146681 | 110747 |
| | | N (SEQ ID NO: 26) | | | | | |
| N(SEQ ID | G1 | Vehicle | 25 | 25 | 25 | 25 | 32 |
| NO: 26) | G2 | SK-RBD (SEQ ID NO: 1) | 25 | 25 | 25 | 25 | 25 |
| | G3 | SK-RBD (SEQ ID NO: 1) + | 25 | 245 | 12611 | 52646 | 41609 |
| | | N(SEQ ID NO: 26) | | | | | |
| | G4 | S-Trimer-P2 (SEQ ID NO: 65) | 25 | 25 | 36 | 179 | 242 |
| | G5 | S-Trimer-P2 (SEQ ID NO: 65) + | 25 | 85 | 1604 | 5683 | 5128 |
| | | N (SEQ ID NO: 26) | | | | | |

TABLE 15
Sequence information

| SEQ ID | | |
|---|---|---|
| NO: | Item | Note |
| 1 | Peptide | SK RBD (328-531) |
| | (Recombinant | |
| | antigen) | |
| 2 | Peptide | Human albumin signal peptide |
| 3 | Peptide | P2 domain |
| 4 | Peptide | Foldon domain |
| 5 | Peptide | Linking signal peptide to SEQ ID NO: 1 |
| 6 | Peptide | RBD-ex1 (321-545) |
| | (Recombinant | |
| | antigen) | |
| 7 | Peptide | RBD-ex2 (321-591) |
| | (Recombinant | |
| | antigen) | |
| 8 | Peptide | RBD-ex3 (321-537) |
| | (Recombinant | |
| | antigen) | |
| 9 | Peptide | SK RBD-P2 |
| | (Recombinant | |
| | antigen) | |
| 10 | Peptide | RBD-ex1-P2 |
| | (Recombinant | |
| | antigen) | |
| 11 | Peptide | RBD-ex2-P2 |
| | (Recombinant | |
| | antigen) | |
| 12 | Peptide | RBD-ex3-P2 |
| | (Recombinant | |
| | antigen) | |
| 13 | Peptide | RBD-Foldon-P2 |
| | (Recombinant | |
| | antigen) | |
| 14 (BEVS) | Polynucleotide | Construct for expression of SEQ ID NO: |
| | | 1 in BEVS |
| 15 (CHO) | Polynucleotide | Construct for expression of SEQ ID NO: |
| | | 1 in CHO |
| 16 (BEVS) | Polynucleotide | Construct for expression of SEQ ID NO: |
| | | 9 in BEVS |
| 17 (CHO) | Polynucleotide | Construct for expression of SEQ ID NO: |
| | | 9 in CHO |
| 18 (BEVS) | Polynucleotide | Construct for expression of SEQ ID NO: |
| | | 10 in BEVS |
| 19 (CHO) | Polynucleotide | Construct for expression of SEQ ID NO: |
| | | 10 in CHO |
| 20 (BEVS) | Polynucleotide | Construct for expression of SEQ ID NO: |
| | | 11 in BEVS |
| 21 (CHO) | Polynucleotide | Construct for expression of SEQ ID NO: |
| | | 11 in CHO |
| 22 (BEVS) | Polynucleotide | Construct for expression of SEQ ID NO: |
| | | 12 in BEVS |
| 23 (CHO) | Polynucleotide | Construct for expression of SEQ ID NO: |
| | | 12 in CHO |
| 24 (BEVS) | Polynucleotide | Construct for expression of SEQ ID NO: |
| | | 13 in BEVS |
| 25 (CHO) | Polynucleotide | Construct for expression of SEQ ID NO: |
| | | 13 in CHO |
| 26 | Peptide | N protein |
| 27 | Peptide | N protein (SP linkage) |
| | (Recombinant | |
| | antigen) | |
| 28 (BEV) | Polynucleotide | Construct for expression of |
| | | N protein in BEVS |
| 29 (CHO) | Polynucleotide | Construct for expression of N protein |
| | | in CHO |
| 30 | Polynucleotide | Spike full length |
| 31 | Polynucleotide | S1 domain |
| 32 | Polynucleotide | S2 domain |
| 33 | Polynucleotide | RBD domain |
| 34 | Peptide | Spike full length |
| 35 | Peptide | S1 domain |
| 36 | Peptide | S2 domain |
| 37 | Peptide | RBD domain |
| 38 (BEVS) | Polynucleotide | Construct for expression of SEQ ID NO: |
| | | 6 in BEVS |
| 39 (CHO) | Polynucleotide | Construct for expression of SEQ ID NO: |
| | | 6 in CHO |
| 40 (BEVS) | Polynucleotide | Construct for expression of SEQ ID NO: |
| | | 7 in BEVS |
| 41 (CHO) | Polynucleotide | Construct for expression of SEQ ID NO: |
| | | 7 in CHO |
| 42 (BEVS) | Polynucleotide | Construct for expression of SEQ ID NO: |
| | | 8 in BEVS |
| 43 (CHO) | Polynucleotide | Construct for expression of SEQ ID NO: |
| | | 8 in CHO |
| 44 | Peptide | Spike protein sequence of |
| | | original Wuhan strain |
| 45 | Peptide | Spike protein sequence of B.1.1.7 |
| 46 | Peptide | Spike protein sequence of B.1.351 |
| 47 | Peptide | Spike protein sequence of B.1.1.248 |
| 48 | Peptide | Spike protein sequence of B.1.429 |
| 49 (CHO) | Polynucleotide | RBD ext1-P2_B.1.1.7 |
| | | (CHO codon optimization) |
| 50 (CHO) | Polynucleotide | RBD ext1-P2_B.1.351(CHO codon |
| | | optimization) |
| 51 (CHO) | Polynucleotide | RBD ext1-P2_B.1.1.248(CHO codon |
| | | optimization) |
| 52 (CHO) | Polynucleotide | RBD ext1-P2_B.1.429(CHO codon |
| | | optimization) |
| 53 (CHO) | Polynucleotide | RBD ext3-foldon-P2_B.1.1.7(CHO codon |
| | | optimization) |
| 54 (CHO) | Polynucleotide | RBD ext3-foldon-P2_B.1.351 (CHO codon |
| | | optimization) |
| 55 (CHO) | Polynucleotide | RBD ext3-foldon-P2_B.1.1.248(CHO codon |
| | | optimization) |
| 56 (CHO) | Polynucleotide | RBD ext3-foldon-P2_B.1.429(CHO codon |
| | | optimization) |
| 57 (CHO) | Polynucleotide | RBD ext1-P2_B.1.1.7 |
| | | (CHO codon optimization) |
| 58 (CHO) | Polynucleotide | RBD ext1-P2_B.1.351(CHO codon |
| | | optimization) |
| 59 (CHO) | Polynucleotide | RBD ext1-P2_B.1.1.248(CHO codon |
| | | optimization) |
| 60 (CHO) | Polynucleotide | RBD ext1-P2_B.1.429(CHO codon |
| | | optimization) |
| 61 (BEVS) | Polynucleotide | RBD-ext3-foldon-P2_B.1.1.7(Insect codon |
| | | optimization) |
| 62 (BEVS) | Polynucleotide | RBD-ext3-foldon-P2_B.1.351 (Insect codon |
| | | optimization) |
| 63 (BEVS) | Polynucleotide | RBD-ext3-foldon-P2_B.1.1.248(Insect codon |
| | | optimization) |
| 64 (BEVS) | Polynucleotide | RBD-ext3-foldon-P2_B.1.429(Insect codon |
| | | optimization) |
| 65 | Polypeptide | SK-S-trimer-P2 recombinant antigen protein |
| 66 (CHO) | Polynucleotide | CHO codon optimization of SK-S-trimer-P2 |
| 67 (BEVS) | Polynucleotide | BEV codon optimization of SK-S-trimer-P2 |

(CHO) refers to a polynucleotide optimized for the CHO expression system, (BEVS) refers to a polynucleotide optimized for the BEVS expression system, and those are represented by _CHO and _BEVS, respectively, in the sequence list.

### INDUSTRIAL APPLICABILITY

The present invention can prevent COVID-19 infection. The present invention can be used as a vaccine.

This application contains references to amino acid sequences and/or nucleic acid sequences which have been submitted concurrently herewith as the sequence listing text file entitled “000072usnp_SequenceListing.TXT”, file size 183 kilobytes (KB), created on 26 Oct. 2022. The aforementioned sequence listing is hereby incorporated by reference in its entirety pursuant to 37 C.F.R. § 1.52(e)(5).

1.-32. (canceled)

33. A recombinant protein for preventing or treating infection of SARS-Coronavirus-2 comprising a polypeptide that forms an extended receptor binding domain (RBD) of a spike protein of SARS-Coronavirus-2.

34. The recombinant protein according to claim 33, wherein a polypeptide forming a P2 domain of Tetanus toxin is optionally linked to the N-terminus or C-terminus of the extended receptor binding domain.

35. The recombinant protein according to claim 34, wherein a polypeptide forming the foldon domain of SEQ ID NO: 4 is linked between the polypeptide forming the extended receptor binding domain and the polypeptide forming the P2 domain of Tetanus toxin.

36. The recombinant protein according to claim 34, wherein the P2 domain of Tetanus toxin and the N-terminus or C-terminus of the extended receptor binding domain is linked by a linker consisting of at least three or more polypeptides.

37. The recombinant protein according to claim 33, wherein the polypeptide that forms an extended receptor binding domain of a spike protein of SARS-Coronavirus-2 contain the wild type RBD polypeptide sequence of SEQ ID NO: 33, and at least 5 to 25 optional polypeptide sequences are added to the polypeptide in the C-terminal and N-terminal directions.

38. The recombinant protein according to claim 33, wherein the recombinant protein is any one polypeptide selected from the group consisting of SEQ ID NOs: 1, 6 to 13, and 65.

39. A gene construct for producing a recombinant protein antigen for preventing or treating infection of SARS-Coronavirus-2, which comprises an open reading frame containing a polynucleotide sequence encoding the recombinant protein according to claim 33.

40. The gene construct according to claim 39, wherein a polynucleotide encoding a heterogonous signal peptide is sequentially operably linked to the open reading frame.

41. The gene construct according to claim 40, wherein a polynucleotide encoding a P2 domain of Tetanus toxin is linked so that the polynucleotides encoding each of the heterologous signal peptide, the open reading frame, and the P2 domain of Tetanus are linked.

42. The gene construct according to claim 41, wherein a polynucleotide encoding a foldon domain is further linked between the polynucleotides encoding the extended receptor binding domain and the P2 domain of Tetanus toxin.

43. The gene construct according to claim 41, wherein the linkage is linked by a polynucleotide encoding a linker consisting of at least three polypeptides.

44. The gene construct according to claim 39, wherein the gene construct consists of any one polynucleotide selected from the group consisting of SEQ ID NOs: 14 to 25, 49 to 64, 66, and 67.

45. A method for preventing or treating infection of SARS-Coronavirus-2, comprising: administering a subject in need thereof a therapeutically effective amount of the antigen protein according to claim 33.

46. The method according to claim 45, which further comprises administering a polypeptide forming any one SARS-Coronavirus-2 derived protein selected from the group consisting of the nucleocapsid (N) protein of SARS-Coronavirus-2 of SEQ ID NO: 26, a matrix (M) protein, and a small envelope (E) protein; an immunological adjuvant; or a mixture thereof.

47. The method according to claim 45, wherein i) a polypeptide forming the recombinant protein and ii) a polypeptide forming the N protein, or its fragments or analogues, and the mixing ratio of the polypeptides (recombinant protein:N protein) are administered in a weight ratio of 1:1 to 500.

48. The method according to claim 46, wherein the immunological adjuvant contains aluminum hydroxide, CpG oligodeoxynucleotide or a mixture thereof.
